(19) United States
(12) Patent Application Publication
(10) Pub. No.: US 2023/0348613 A1
(43) Pub. Date: Nov. 2, 2023

(54) METHODS AND ANTIBODIES IN TREATMENT OF FOCAL SEGMENTAL GLOMERULOSCLEROSIS (FSGS)

(71) Applicant: The Regents of the University of California, Oakland, CA (US)

(72) Inventors: Charles S. Craik, San Francisco, CA (US); Flavio Vincenti, San Francisco, CA (US)

(21) Appl. No.: 17/998,641

(22) PCT Filed: Jun. 3, 2021
(86) PCT No.: PCT/US2021/035743

§ 371 Date: Nov. 11, 2022

Related U.S. Application Data
(60) Provisional application No. 63/035,426, filed on Jun. 5, 2020.

(51) Int. Cl. C07K 16/28; A61P 13/12

(52) U.S. Cl. CPC C07K 16/2896; A61P 13/12; A61K 2039/505

(57) ABSTRACT

The present disclosure provides methods of use of antibodies that specifically bind to urokinase plasminogen activator receptor (uPAR/CD87), as well as to is its soluble counterpart, soluble uPAR (suPAR), in treatment or prevention of focal segmental glomerulosclerosis (FSGS).

### CROSS-REFERENCE

This application claims the benefit of U.S. Provisional Application Ser. No. 63/035,426, filed Jun. 5, 2020, the disclosure of which application is herein incorporated by reference in its entirety.

### STATEMENT REGARDING FEDERALLY SPONSORED RESEARCH

This invention was made with government support under Grant No. CA196276 awarded by the National Institutes of Health. The government has certain rights in the invention.

### INTRODUCTION

Focal segmental glomerulosclerosis (FSGS) is a kidney disease in which scar tissue develops on the glomeruli, which are responsible for filtering waste from the blood. FSGS, which is characterized by nephrotic range proteinuria and kidney dysfunction, can lead to kidney failure, which in turn requires treatment by either dialysis or kidney transplant.

FSGS accounts for 20% of cases of nephrotic syndrome, hand as an incidence of 7 per million and a prevalence of 5% of patients with end stage kidney disease. About 30% of FSGS patients experience a recurrence of FSGS after transplantation, and usually within hours, days and weeks of the transplant. Recurrent FSGS (rFSGS) is an important cause of graft loss after kidney transplantation.

Current therapies for treatment or prevention of FSGS are non-specific. Native kidney FSGS is treated with steroids, calcineurin inhibitors and rituximab with suboptimal results. Recurrent FSGS is treated with plasmapheresis, but few patients have long lasting remission. Both native kidney FSGS and its recurrence after transplantation represents unmet needs.

### SUMMARY

The present disclosure provides methods of use of antibodies that specifically bind to urokinase plasminogen activator receptor (uPAR/CD87), as well as to is its soluble counterpart, soluble uPAR (suPAR), in treatment or prevention of focal segmental glomerulosclerosis (FSGS).

Accordingly, the present disclosure provides a method of treating or preventing focal segmental glomerulosclerosis (FSGS) in a subject comprising administering to the subject an antibody, or antigen-binding fragment thereof, that competes for binding to uPAR with an antibody, or antigen-binding fragment thereof, comprising a variable heavy chain (VH) polypeptide comprising VH complementarity determining regions (CDRs) of an antibody heavy chain variable region comprising the amino acid sequence QVQLQQSGPGLVKPSQTLSLTCAISGDSVSSNSAAWNWIRQSPSRGLEWLGRTYYRSKWYNDY AVSVKSRIIINPDTSKNQFSLQLNSVTPEDTAVYYCARDPGGPLDDSFDIWGQGTMVTVSSASTK GPSVFPLAPSSKSTSGGTAALGCLVKDYFPEPVTVSWNSGALTSGVHTFPAVLQSSGLYSLSSVV TVPSSSLGTQTYICNVNHKPSNTKVDKKVEPKSC (SEQ ID NO: 1); and a variable light chain (VL) polypeptide comprising VL CDRs of an antibody light chain variable region comprising amino acid sequence

(SEQ ID NO: 2)
MTQSPLSLPVTPGEPASISCRSSQSLLRSNGYNYLDWYLQKPGQSPQLL
IYLGSIRASGVPDRFSGSGSGTDFTLRISRVEAEDVGVYYCMQALQTPF
TFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYAC
EVTHQGLSSPVTKSFNRGEC

In related aspects, the antibody, or antigen-binding fragment thereof, administered in the method comprises a VH CDR1 comprising the amino acid sequence of GDSVSSNSAAWN (SEQ ID NO: 3); a VH CDR2 comprising the amino acid sequence of RTYYRSKWYND (SEQ ID NO: 4); a VH CDR3 comprising the amino acid sequence of DPGGPLDDSFDI (SEQ ID NO: 5); a VL CDR1 comprising the amino acid sequence of RSSQSLLRSNGYNYLD (SEQ ID NO: 6); a VL CDR2 comprising the amino acid sequence of LGSIRAS (SEQ ID NO:7); and a VL CDR3 comprising the amino acid sequence of MQALQTPFT (SEQ ID NO: 8).

In related aspects, the antibody, or antigen-binding fragment thereof, administered in the method comprises a) a heavy chain polypeptide comprising an amino acid sequence of at least 85% amino acid sequence identity to the full length VH of 2G10; and b) a light chain polypeptide comprising an amino acid sequence of at least 85% amino acid sequence identity to the full length VL of 2G10.

In related aspects of any of the above methods, the subject is at risk of FSGS, and said administering is effective to prevent or ameliorate FSGS in the subject. In related aspects of any of the above methods, the subject has or is suspected of having FSGS, and said administering is effective to treat FSGS in the subject. In related aspects of any of the above methods, the subject is a candidate for kidney transplant or has undergone a kidney transplant. In related aspects of any of the above methods, the subject has undergone a kidney transplant and is at risk of recurrent FSGS.

In related aspects of any of the above methods, the subject is a kidney transplant candidate and the antibody, or antigen-binding fragment thereof, is administered prior to kidney transplant, at the time of kidney transplant, or following kidney transplant.

The present disclosure also provides methods of inhibiting activity of urokinase-type plasminogen activator receptor (uPAR) and/or soluble uPAR (suPAR) in a subject having detectable blood level of suPAR, the method comprising administering to the subject an effective amount of an antibody, or antigen-binding fragment thereof, that specifically binds uPAR and suPAR, wherein the antibody, or antigen-binding fragment thereof, competes for binding to uPAR with an antibody, or antigen-binding fragment thereof, comprising: a variable heavy chain (VH) polypeptide comprising VH complementarity determining regions (CDRs) of an antibody heavy chain variable region comprising the amino acid sequence QVQLQQSGPGLVKPSQTLSLTCAISGDSVSSNSAAWNWIRQSPSRGLEWLGRTYYRSKWYNDY AVSVKSRIIINPDTSKNQFSLQLNSVTPEDTAVYYCARDPGGPLDDSFDIWGQGTMVTVSSASTK GPSVFPLAPSSKSTSGGTAALGCLVKDYFPEPVTVSWNSGALTSGVHTFPAVLQSSGLYSLSSVV TVPSSSLGTQTYICNVNHKPSNTKVDKKVEPKSC (SEQ ID NO: 1); and a variable light chain (VL) polypeptide comprising VL CDRs of an antibody light chain variable region comprising amino acid sequence

(SEQ ID NO: 2)
MTQSPLSLPVTPGEPASISCRSSQSLLRSNGYNYLDWYLQKPGQSPQLL
IYLGSIRASGVPDRFSGSGSGTDFTLRISRVEAEDVGVYYCMQALQTPF
TFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYAC
EVTHQGLSSPVTKSFNRGEC

In related aspects, the antibody, or antigen-binding fragment thereof, administered in the method comprises a VH CDR1 comprising the amino acid sequence of GDSVSSNSAAWN (SEQ ID NO: 3); a VH CDR2 comprising the amino acid sequence of RTYYRSKWYND (SEQ ID NO: 4); a VH CDR3 comprising the amino acid sequence of DPGGPLDDSFDI (SEQ ID NO: 5); a VL CDR1 comprising the amino acid sequence of RSSQSLLRSNGYNYLD (SEQ ID NO: 6); a VL CDR2 comprising the amino acid sequence of LGSIRAS (SEQ ID NO:7); and a VL CDR3 comprising the amino acid sequence of MQALQTPFT (SEQ ID NO: 8).

In related aspects, the antibody, or antigen-binding fragment thereof, administered in the method comprises a) a heavy chain polypeptide comprising an amino acid sequence of at least 85% amino acid sequence identity to the full length VH of 2G10; and b) a light chain polypeptide comprising an amino acid sequence of at least 85% amino acid sequence identity to the full length VL of 2G10.

In related aspects of any of the above methods, the subject is at risk of FSGS, and said administering is effective to prevent or ameliorate FSGS in the subject. In related aspects of any of the above methods, the subject has or is suspected of having FSGS, and said administering is effective to treat FSGS in the subject. In related aspects of any of the above methods, the subject is a candidate for kidney transplant or has undergone a kidney transplant. In related aspects of any of the above methods, the subject has undergone a kidney transplant and is at risk of recurrent FSGS.

In related aspects of any of the above methods, the subject is a kidney transplant candidate and the antibody, or antigen-binding fragment thereof, is administered prior to kidney transplant, at the time of kidney transplant, or following kidney transplant.

### BRIEF DESCRIPTION OF FIGURES

FIG. 1 shows the results of incubation of pretransplant sera from recurrent FSGS (rFSGS) patients on a human podocyte cell model. Pretransplantation sera from rFSGS patients cause loss of stress fibers, an effect which was not affected by addition of a control IgG antibody.

FIG. 2 shows the effect of pretransplant sera from recurrent FSGS (rFSGS) patients on a human podocyte cell model in the absence or presence of the anti-uPAR antibody 2G10. T

### DETAILED DESCRIPTION OF EMBODIMENTS

The present disclosure provides methods of use of antibodies that specifically bind to urokinase plasminogen activator receptor (uPAR/CD87), particularly its soluble counterpart, soluble uPAR, in treatment of focal segmental glomerulosclerosis (FSGS), including recurrent FSGS (rFSGS).

In general, antibodies that find use in the methods of the present disclosure include those that bind specifically bind uPAR, as well as its soluble counterpart, soluble uPAR (suPAR), and inhibit ligand binding. Without being held to theory, the anti-uPAR antibodies for use in the methods of the present disclosure are those that inhibit binding of suPAR in patient serum to integrin on the surface of human podocytes. Without being held to theory, binding of suPAR to integrins on the surface of human podocytes leads to downstream signaling that results in disruption of stress fibers, leading to podocyte effacement and proteinuria.

The data presented herein support the application of such anti-uPAR/anti-suPAR antibodies that disrupt uPAR/suPAR ligand binding in methods and compositions, including the diagnosis of subjects at risk of FSGS, particularly recurrent FSGS, and treatment of FSGS, including primary idiopathic FSGS, primary non-idiopathic FSGS, and secondary FSGS.

Compositions comprising an antibody that bind uPAR and its soluble form (suPAR) or antigen-binding fragment thereof, for use in a method of treatment as disclosed herein are also contemplated.

Before the present invention and specific embodiments of the invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. That the upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials are now described. All publications mentioned herein are incorporated herein by reference to disclose and describe the methods and/or materials in connection with which the publications are cited.

It must be noted that as used herein and in the appended claims, the singular forms “a”, “an,” and “the” include plural referents unless the context clearly dictates otherwise. Thus, for example, reference to “an antigen” includes a plurality of such antigens and reference to “the peptide” includes reference to one or more peptides and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

When describing the compositions, pharmaceutical formulations containing such, and methods of producing and using such compositions, the following terms have the following meanings unless otherwise indicated. It should also be understood that any of the moieties defined forth below may be substituted with a variety of substituents, and that the respective definitions are intended to include such substituted moieties within their scope. Other terms are defined within the specification, e.g., infra.

The terms “polypeptide” and “protein” are used interchangeably throughout the application and mean at least two covalently attached amino acids, which includes proteins, polypeptides, oligopeptides, peptides, and fragments thereof. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. Normally, the amino acids are in the (S) or L-configuration, except for glycine. If non-naturally occurring side chains are used, non-amino acid substituents may be used, for example to prevent or retard in vivo degradation. Naturally occurring amino acids may be used and the protein may be a cellular protein that is either endogenous or expressed recombinantly. In some cases, the proteins of the present invention may be synthesized using any protein in vivo or in vitro protein synthesis technique understood in the art. The terms “polypeptide” and “protein” include fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like. Polypeptides may be of any size, and the term “peptide” refers to polypeptides that are 5-50 residues (e.g., 8-20 residues) in length. In some cases, proteins may be modified by covalent or non-covalent attachment of other peptide or non-peptide molecules including but not limited to one or more molecules or compositions comprised of fluorescent dyes, polyethylene glycol or other polymer, biotin, enzymes, radionuclides, MRI contrast agents, therapeutics, or chemotherapeutics as described in more detail below.

By “nucleic acid” herein is meant either DNA or RNA, or molecules which contain both deoxy- and ribonucleotides. Nucleic acid may be naturally occurring or synthetically made, and as such, includes analogs of naturally occurring polynucleotides in which one or more nucleotides are modified over naturally occurring nucleotides.

The term, “endogenous”, as used herein, refers to biomolecules, such as proteins, that are naturally-occurring within an organism.

The term “carrier” as used in the context of a carrier conjugated to an antibody includes a peptide or protein carrier, a non-peptide or protein carrier (e.g. a non-peptide polymer).

The term “cell surface antigen” (or “cell surface epitope”) refers to an antigen (or epitope) on surface of a cell that is extracellularly accessible during at least one cell cycle or developmental stage of the cell, including antigens that are extracellularly accessible during all stages of the cell cycle. “Extracellularly accessible” in this context refers to an antigen that can be bound by an antibody provided outside the cell without need for permeabilization of the cell membrane.

The term “conjugated” generally refers to a chemical linkage, either covalent or non-covalent, usually covalent, that proximally associates one molecule of interest with second molecule of interest.

The terms “antigen” and “epitope” are well understood in the art and refer to the portion of a macromolecule (e.g., a polypeptide) which is specifically recognized by a component of the immune system, e.g., an antibody. As used herein, the term “antigen” encompasses antigenic epitopes, e.g., fragments of an antigen which are antigenic epitopes. Epitopes can be recognized by antibodies in solution, e.g. free from other molecules.

The term “effective amount” of a composition as provided herein is intended to mean a non-lethal but sufficient amount of the composition to provide the desired utility. For instance, for eliciting a favorable response in a subject to treat a disorder (e.g., FSGS), the effective amount is the amount which eliminates or diminishes the symptoms associated with the disorder, e.g., so as to provide for treatment or prevention of FSGS, e.g., rFSGS. As will be pointed out below, the exact amount required will vary from subject to subject, depending on the species, age, and general condition of the subject, the severity of the condition or disease that is being treated, the particular composition used, its mode of administration, and the like. An appropriate effective amount may be determined by one of ordinary skill in the art using only routine experimentation.

The term “in combination with” as used herein refers to uses where, for example, a first therapy is administered during the entire course of administration of a second therapy; where the first therapy is administered for a period of time that is overlapping with the administration of the second therapy, e.g. where administration of the first therapy begins before the administration of the second therapy and the administration of the first therapy ends before the administration of the second therapy ends; where the administration of the second therapy begins before the administration of the first therapy and the administration of the second therapy ends before the administration of the first therapy ends; where the administration of the first therapy begins before administration of the second therapy begins and the administration of the second therapy ends before the administration of the first therapy ends; where the administration of the second therapy begins before administration of the first therapy begins and the administration of the first therapy ends before the administration of the second therapy ends. As such, “in combination” can also refer to regimen involving administration of two or more therapies. “In combination with” as used herein also refers to administration of two or more therapies which may be administered in the same or different formulations, by the same or different routes, and in the same or different dosage form type.

The term “isolated” is intended to mean that a compound is separated from all or some of the components that accompany it in nature. “Isolated” also refers to the state of a compound separated from all or some of the components that accompany it during manufacture (e.g., chemical synthesis, recombinant expression, culture medium, and the like).

The term “antibody” refers to a polypeptide having complementarity determining regions (CDRs) that confer specific binding affinity of the polypeptide for an antigen, e.g, suPAR, uPAR. “Antibody” encompasses polyclonal and monoclonal antibody preparations where the antibody may be of any class of interest (e.g., IgM, IgG, and subclasses thereof), as well as preparations including hybrid antibodies, altered antibodies, covalently modified antibodies, F(ab′)₂ fragments, F(ab) molecules, Fv fragments, single chain fragment variable displayed on phage (scFv), single chain antibodies (e.g. single-chain Fab), single domain antibodies, affibodies, diabodies, chimeric antibodies, human antibodies, humanized antibodies, and functional fragments thereof which exhibit immunological binding properties of the parent antibody molecule. The antibodies described herein may be detectably labeled, e.g., with a radioisotope, an enzyme which generates a detectable product, a fluorescent protein, and the like. The antibodies may be further conjugated to other moieties, members of specific binding pairs, e.g., biotin (member of biotin-avidin specific binding pair), and the like. The antibodies may also be bound to a support (e.g., a solid support), such as a polystyrene plate or bead, test strip, and the like.

Antibodies can include the kappa and lambda light chains and the alpha, gamma (IgG₁, IgG₂, IgG₃, IgG₄), delta, epsilon and mu heavy chains or equivalents in other species. Full-length immunoglobulin “light chains” (usually of about 25 kDa or about 214 amino acids) comprise a variable region of about 110 amino acids at the NH₂-terminus and a kappa or lambda constant region at the COOH-terminus. Full-length immunoglobulin “heavy chains” (of about 50 kDa or about 446 amino acids), similarly comprise a variable region (of about 116 amino acids) and one of the aforementioned heavy chain constant regions, e.g., gamma (of about 330 amino acids).

Light or heavy chain variable regions are generally composed of a series of “framework” regions (FRs) flanking three hypervariable regions, also called CDRs. The extent of the framework regions and CDRs have been precisely defined (see, “Sequences of Proteins of Immunological Interest,” E. Kabat et al., U.S. Department of Health and Human Services, 1991, and Lefranc et al. IMGT, the international ImMunoGeneTics information System®. Nucl. Acids Res., 2005, 33, D593-D597)). A detailed discussion of the Kabat numbering system is provided on the World Wide Web at kabatdatabase.com/index.html. CDR and framework sequences may also be defined by the Chothia numbering system. The sequences of the framework regions of different light or heavy chains are relatively conserved within a species. The framework region of an antibody, that is the combined framework regions of the constituent light and heavy chains, serves to position and align the CDRs. The CDRs are primarily responsible for binding to an epitope of an antigen.

The term “monoclonal antibody” refers to an antibody composition having a homogeneous antibody population. The term is not limited by the manner in which it is made. The term encompasses whole immunoglobulin molecules, as well as Fab molecules, F(ab′)2 fragments, Fv fragments, single chain fragment variable displayed on phage (scFv), fusion proteins comprising an antigen-binding portion of an antibody and a non-antibody protein, and other molecules that exhibit binding properties of the parent monoclonal antibody molecule. Methods of making monoclonal antibodies are known in the art.

The term “specific binding of an antibody” or “antigen-specific antibody” in the context of a characteristics of an antibody refers to the ability of an antibody to preferentially bind to a particular antigen that is present in a homogeneous mixture of different antigens. Where the epitope bound on the antigen is present on different forms of the same antigen (e.g., on both uPAR and suPAR), “specific binding of an antibody” or “antigen-specific antibody” refers to the ability of the antibody to preferentially bind to either form of the particular antigen having that epitope (e.g, either uPAR or suPAR) that is present in a homogeneous mixture of different antigens. In certain embodiments, a specific binding interaction will discriminate between desirable and undesirable antigens (or “target” and “non-target” antigens) in a sample, in some embodiments more than about 10 to 100-fold or more (e.g., more than about 1000- or 10,000-fold). The affinity between an antibody and antigen when they are specifically bound in an antibody-antigen complex can be characterized by a K_{D} (dissociation constant) of less than 10⁻⁶ M, less than 10⁻⁷ M, less than 10⁻⁸ M, less than 10⁻⁹ M, less than 10⁻⁹ M, less than 10⁻¹¹ M, or less than about 10¹² M or less.

“Conservative amino acid substitution” refers to a substitution of one amino acid residue for another sharing chemical and physical properties of the amino acid side chain (e.g., charge, size, hydrophobicity/hydrophilicity). “Conservative substitutions” are intended to include but are not limited to substitution within the following groups of amino acid residues: gly, ala; val, ile, leu; asp, glu; asn, gln; ser, thr; lys, arg; and phe, tyr. Conservative amino acid substitutions in the context of an antibody disclosed herein are selected so as to preserve the interaction between the antibody and the protease of interest. Other conservative substitutions that can preserve size, chemical property, and/or shape includes val, thr; asp, asn, glu, gln; leu, phe, tyr, trp; lys, leu; trp, phe, and tyr; and ala, val, tyr.

The term “pharmaceutically acceptable” refers to a material that is not biologically or otherwise undesirable, i.e., the material is of a medically acceptable quality and composition that may be administered to an individual along with the selected active pharmaceutical ingredient without causing any undesirable biological effects or interacting in a deleterious manner with any of the other components of the pharmaceutical composition in which it is contained.

The term “pharmaceutically acceptable excipient” as used herein refers to any suitable substance which provides a pharmaceutically acceptable vehicle for administration of a compound(s) of interest to a subject. “Pharmaceutically acceptable excipient” can encompass substances referred to as pharmaceutically acceptable diluents, pharmaceutically acceptable additives and pharmaceutically acceptable carriers.

The term “purified” is intended to mean a compound of interest has been separated from components that accompany it in nature and provided in an enriched form. “Purified” also refers to a compound of interest separated from components that can accompany it during manufacture (e.g., in chemical synthesis, recombinant expression, culture medium, and the like) and provided in an enriched form. Typically, a compound is substantially pure when it is at least 50% to 60%, by weight, free from organic molecules with which it is naturally associated or with which it is associated during manufacture. Generally, the preparation is at least 75%, more usually at least 90%, and generally at least 99%, by weight, of the compound of interest. A substantially pure compound can be obtained, for example, by extraction from a natural source (e.g., bacteria), by chemically synthesizing a compound, or by a combination of purification and chemical modification. A substantially pure compound can also be obtained by, for example, enriching a sample having a compound that binds an antibody of interest. Purity can be measured by any appropriate method, e.g., chromatography, mass spectroscopy, HPLC analysis, polyacrylamide gel electrophoresis, etc.

The terms “subject,” “host,” “patient,” and “individual” are used interchangeably herein to refer to any mammalian subject for whom diagnosis or therapy is desired, particularly humans. Other subjects may include cattle, dogs, cats, guinea pigs, rabbits, rats, mice, horses, and so on.

In the context of FSGS therapies and diagnostics described herein, “subject” or “patient” is used interchangeably herein to refer to a subject having, suspected of having, or at risk of developing recurrent FSGS. In some cases, the subject is one who is a candidate for, or who has undergone, a kidney transplant. Samples obtained from such subject, particularly blood or blood-derived samples expected to contain suPAR if present, are likewise suitable for use in the diagnostic methods of the present disclosure to identify subjects amenable to anti-suPAR/uPAR therapy. As used herein, the terms “determining,” “cmeasuring,” and “assessing,” and “assaying” are used interchangeably and include both quantitative and qualitative determinations.

It is further noted that the claims may be drafted to exclude any optional or alternative element. As such, this statement is intended to serve as antecedent basis for use of such exclusive terminology as “solely”, “only” and the like in connection with the recitation of claim elements, or the use of a “negative” limitation.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed. To the extent a definition of a term set out in a document incorporated herein by reference conflicts with the definition of a term explicitly defined herein, the definition set out herein controls.

Examples of methods and compositions employable therein are described first in greater detail, followed by a review of the various specific compositions, formulations, kits and the like that may find use in the methods of the present disclosure, as well as a discussion of representative applications in which the methods and compositions of the present disclosure find use.

### Upar and Supar Binding Agents

The present disclosure provides uPAR-binding agents (e.g. anti-uPAR antibodies, also referred to as “uPAR antibodies”), and suPAR-binding agents (e.g., anti-suPAR antibodies). Antibodies that find use in the methods and compositions of the present disclosure are antibodies that specifically bind an epitope of uPAR that is present on suPAR, especially an epitope that, when bound, inhibits ligand binding to uPAR, ligand binding to suPAR, or both. Thus, an antibody may bind to both cell surface uPAR and to suPAR and be regarded as a uPAR-specific antibody (or “anti-uPAR antibody”) as well as a suPAR-specific antibody (or “anti-suPAR antibody”). Such antibodies that specifically bind both the cell surface (uPAR) and soluble (suPAR) forms of the receptor may be referred to herein as anti-uPAR/suPAR antibodies or at anti-suPAR/uPAR antibodies.

Where the agent is an antibody, the antibody includes a whole antibody (e.g. IgG), an antigen-binding fragment thereof, single-chain Fabs, single chain Fv (e.g. diabodies or V_{H}H), Fab′2, minibody, and synthetic uPAR antibody that comprise portions of an antibody.

“uPAR” is also known as urokinase plasminogen activator receptor, urokinase receptor, uPA receptor, or CD87 (Cluster of Differentiation 87). uPAR is composed of three different domains of the Ly-6/uPAR/alpha-neurotoxin family. All three domains are involved in high affinity binding of the primary ligand, urokinase. Besides the primary ligand urokinase, uPAR interacts with several other proteins, including vitronectin, the uPAR associated protein (uPARAP) and the integrin family of membrane proteins. Upon binding of uPA to its receptor (uPAR) it mediates various cellular activities such as adhesion, migration, differsentiation, and proliferation. In podocytes, uPAR is one of the pathways capable of activating αvρ3 integrin promoting cell motility and activation of small GTPases, such as Cdc42 and Rac1, which can lead to podocyte contraction, shifting from a stationary to motile phenotype and leading to foot process effacement and proteinuria.

As used herein, “uPAR” refers to urokinase plasminogen activator receptor, including those whose amino acid sequences that are at least about 80%, at least about 85%, at least about 90%, at least about 95%, at least about 98%, at least about 99%, or 100% identical to the amino acid sequence of a naturally-occurring allelic variant and/or isoform thereof. Many mammalian uPARs and their corresponding isoforms are known in the art. For example, the amino acid sequence of the longest human isoform is available as GenBank Accession No. NP_002650.1 and UniProt Accession No. Q03405.

Binding of ligands and/or integrins to uPAR is involved in signaling that can lead to proliferation. Certain signaling cascades that are initiated by activated uPAR mediate the regulation of cellular shape, adhesion, and mobility, and thus play a role in cell invasion. Accordingly, preventing ligands such as uPA and/or integrins (e.g. β1 integrins, such as α5β1 or α3 ρ1) from binding to uPAR can reduce the effects of proliferative signaling cascades and those signals leading to angiogenesis. A subject binding agent can exhibit features that allow not only competitive binding with proteins (e.g. integrins and/or ligands) that bind to uPAR and/or suPAR, but also potent inhibition of uPAR- and/or suPAR-mediated cell signaling.

The uPAR- and suPAR-binding agents of the present disclosure can find use in a variety of applications, including use in various methods of treating a host suffering from a disease or condition associated with uPAR signaling, as well as in diagnosis of various diseases and conditions associated with uPAR expression. For example, a subject agent, such as an antibody, is specific for the integrin-binding site on uPAR and suPAR and may be used to prevent or treat uPAR- and/or suPAR-mediated disease, such as FSGS. Uses of a subject agent will be described later.

“suPAR” or “soluble uPAR” as used herein refers to a soluble form of uPAR, which is released from uPAR expressing cells. Full-length suPAR (suPARI-III) can be cleaved into another two soluble forms with different biologic properties, suPARII-III and suPARI. The I-III portion of suPAR can compete with uPARI-III for uPA binding. suPAR can be found in various body fluids including blood, plasma, serum, urine, saliva, and cerebrospinal fluid (CSF) in different concentrations and exhibits similar functions as uPAR.

suPAR and/or uPAR-expressing cells can serve as targets for the uPAR antibodies of the present disclosure. For example, uPAR-binding agents (e.g. antibodies) of the present disclosure can be used to bind human cells that express surface exposed uPAR. The binding may be specific such that cells that express uPAR are bound by the subject antibody, but cells that do not express uPAR are not detectbly bound by the subject antibody. The binding may be specific so suPAR is boudn by the subject antibody, but other antigens present in the circulatory system of a subject (e.g., in the blood of a subject) are not detectably bound. The uPAR expressed in cells, or the suPAR released from uPAR expressing cells, may be endogenous, recombinants, naturally-occurring variants and isoforms, and/or a homolog of human uPAR (murine, rat, bovine, primates, etc.). Particularly, uPAR molecules that are expressed by and, in some instances released as suPAR from, kidney cells, e.g., podocytes, can be bound by the subject antibody.

As a reference, an amino acid sequence of uPAR is provided below and can also be found in RSCB Protein Data Bank identified as 3BT1. Numbering system used in the present disclosure to refer to an amino acid residue position in uPAR would be in the context of the following amino acid sequence:

(SEQ ID NO: 9)
LRCMQCKTNGDCRVEECALGQDLCRTTIVRLWEEGEELELVEKSCTHSE
KTNRTLSYRTGLKITSLTEVVCGLDLCNQGNSGRAVTYSRSRYLECISC
GSSDMSCERGRHQSLQCRSPEEQCLDVVTHWIQEGEEGRPKDDRHLRGC
GYLPGCPGSNGFHNNDTFHFLKCCNTTKCNEGPILELENLPQNGRQCYS
CKGNSTHGCSSEETFLIDCRGPMNQCLVATGTHEPKNQSYMVRGCATAS
MCQHAHLGDAFSMNHIDVSCCTKSGCNHPDLDVQYR

The present disclosure provides uPAR and suPAR agents (e.g. antibodies) that compete with and/or disrupt integrin binding to uPAR. Integrins encompass β1 integrins, such as α5β1 or α3β1. The agents thus find use in inhibiting integrin binding to cells (e.g., human cells expressing uPAR). For example, antibodies of clone 3C6 inhibit α5β1 and α3β1 integrin binding to uPAR. This inhibition may be due to the binding of the antibody to an epitope involved in the interaction between integrin and uPAR (e.g. integrin binding site) or to an epitope outside of the binding site so that uPAR is modified in a way to decrease uPAR's affinity to integrin (e.g. allosteric site). As such, a uPAR and suPAR antibodies of the present disclosure can compete with an antibody that binds to an epitope located in the integrin-binding site (e.g. α5β1 and/or α3β1 integrin binding site).

The present disclosure also provides agents that compete with and/or inhibit uPA binding to uPAR. Urokinase-type plasminogen activator (uPA, also known as urokinase), an endogenous ligand of uPAR, is a member of a family of enzymes that exhibit protease activity described as EC 3.4.21.73 according to the IUMBM enzyme nomenclature. UPAR antibodies can decrease binding of uPA to uPAR by competitive inhibition, where the antibody binds to the same site of uPAR as uPA binds or at a different site outside of the uPA binding site (e.g. allosteric site), or by noncompetitive inhibition. Examples of antibodies that can inhibit uPA binding to uPAR include antibodies from clone 2E9 and antibodies from clone 2G10.

As such, a uPAR or suPAR antibody of the present disclosure can compete with an antibody that binds to an epitope located in the uPA-binding site. One or more epitopes of a uPA-binding site can be found in domain I and/or domain II of uPAR and of suPAR. Domain I corresponds to an amino acid sequence of uPAR from about amino acid residue position 1 to about position 80. Domain II corresponds to an amino acid sequence of uPAR from about amino acid residue position 91 to about position 191.

As noted above, antibody affinity for uPAR or for suPAR may be described by the dissociation constant, K_{D}. Antibodies of the present disclosure, for example, include those having a K_{D} for uPAR of less than about 1000 nM, less than about 500 nM, less than about 300 nM, less than about 200 nM, less than about 100 nM, less than about 80 nM, less than about 60 nM, less than about 55 nM, less than about 50 nM, less than about 40 nM, less than about 30 nM, less than about 25 nM, less than about 20 nM, less than about 10 nM, less than about 5 nM, less than about 2 nM, less than about lnM, less than about 750 pM, less than about 500 pM, less than about 300 pM, less than about 200 pM, less than about 100 pM, or less than about 50 pM. For example, the divalent IgG antibody derived from clone 2G10 has a K_{D} of about 40.5 nM.

uPAR and suPAR antibodies of the present disclosure include antibodies that facilitate a decrease in cellular signaling associated with uPAR ligand or integrin binding. Such antibodies can find use in, for example, decreasing cellular signaling effects caused by binding of ligand to uPAR. Cellular signaling effects can be assessed by, for example, modulation of (e.g., a decrease in) phosphorylation levels of kinases associated with uPAR signaling, such as extracellular signal-regulated kinases (ERKs), mitogen activated kinases (MAPK), and/or microtubule-associated protein kinase. For example, antibodies of the present disclosure include those that can inhibit podocyte depolarization and in turn, inhibit podocyte depolarization, in the presence of suPAR and/or uPAR/suPAR ligands.

Amino Acid Sequences

suPAR and uPAR binding agents of the present disclosure include antibodies that bind an epitope in the ligand-binding region and/or integrin-binding region of uPAR and, thus, of suPAR. Several examples of a subject antibody are described below.

Antibodies of the present disclosure include antibodies having one, two, or three heavy chain CDRs about 85%, 90%, 95%, 98%, 99%, or 100% identical to V_{H} CDR1, V_{H} CDR2, and/or V_{H} CDR3 as described below. Antibodies of the present disclosure include antibodies having one, two, or three light chain CDRs about 85%, 90%, 95%, 98%, 99%, or 100% identical to V_{L} CDR1, V_{L} CDR2, and/or V_{L} CDR3 as described below. All CDRs may be derived from the same antibody or be independently selected from different antibodies described herein.

The V_{H} and V_{L} CDRs are separated by framework regions (FR). Amino acid sequences for FRs are exemplified by the FRs of the uPAR and suPAR antibodies disclosed herein. uPAR and suPAR antibodies include those containing FRs or other linkers having amino acid sequence that are different from the framework regions disclosed herein. Conservative amino acid substitutions may also be contemplated for any amino acid residue of CDR, framework regions, or linker regions. Other substitutions may be contemplated based on alignments of the amino acid sequences of the CDRs, the full-length VH, and/or the full-length VL disclosed herein.

Optional linkers within a heavy chain or light chain polypeptide of an antibody may comprise amino acid residues or non-peptide polymers. The linkers may have a length of from about 1 to about 100 monomers, e.g., from about 2 to about 5, from about 7 to about 10, from about 10 to about 15, from about 15 to about 20, from about 20 to about 25, from about 25 to about 30, from about 30 to about 50, from about 50 to about 75, or from about 75 to about 100 monomers.

Examples of uPAR and suPAR antibodies of the present disclosure include an antibody comprising a heavy chain polypeptide having an amino acid sequence having at least about 80%, at least about 85%, at least about 90%, at least about 95%, at least about 98%, or at least about 99%, or 100% amino acid sequence identity to a contiguous stretch of the amino acid sequence set forth as 2G10 V_{H}.

Examples of uPAR and suPAR antibodies of the present disclosure include an antibody comprising a light chain polypeptide having an amino acid sequence having at least about 85%, at least about 90%, at least about 95%, at least about 98%, or at least about 99%, or 100% amino acid sequence identity to a contiguous stretch of the amino acid sequence set forth as 2G10 V_{L}.

Examples of uPAR and suPAR antibodies of the present disclosure include an antibody comprising a light chain polypeptide comprising one or more CDRs (CDR1, CDR2 or CDR3) of the variable region of a light chain polypeptide as described below and a heavy chain polypeptide comprising one or more CDRs (CDR1, CDR2, or CDR3) of the variable region of any heavy chain polypeptide as described below. One or more amino acid residues in one or more of the CDRs set forth above may be deleted, inserted, or substituted in the subject antibody. Conservative substitutions may also be present.

suPAR and uPAR antibodies of the present disclosure may be of any subclass (e.g. IgG, IgE, IgD, IgA, or IgM). The antibody may be fully human or may be a humanized monoclonal antibody. Chimeric antibodies composed of human and non-human amino acid sequences are also contemplated by the present disclosure. Antibodies of the present disclosure encompass antibodies and antibody fragments that are capable of exhibiting immunological binding properties of the antibodies described herein, e.g., antibodies that compete for binding of an epitope bound by any of the antibodies exemplified herein. Example of antibody fragments include, but are not limited to, Fab, Fab′ and F(ab′)₂, Fd, single-chain Fvs (scFv), single-chain immunoglobulins (e.g., wherein a heavy chain, or portion thereof, and light chain, or portion thereof, are fused), disulfide-linked Fvs (sdFv), diabodies, triabodies, tetrabodies, scFv, affibodies, minibodies, Fab minibodies, and dimeric scFv and any other fragments comprising a V_{L} and a V_{H} domain in a conformation such that a specific antigen binding region is formed. Antibody fragments, including single-chain antibodies, may comprise the variable region(s) alone or in combination with the entire or partial of the following: a heavy chain constant domain, or portion thereof, e.g., a CH1, CH2, CH3, transmembrane, and/or cytoplasmic domain, on the heavy chain, and a light chain constant domain, e.g., a Cₖₐₚₚₐ or C_{lambda} domain, or portion thereof on the light chain. Also included in the present disclosure are any combinations of variable region(s) and CH1, CH2, CH3, Cₖₐₚₚₐ, C_{lambda}, transmembrane and cytoplasmic domains. One or more fragments of the antibody may also be provided as cyclized forms.

The disclosure also provides agents (e.g. antibodies) that are modified by conjugation to a moiety that can provide for a desired characteristic (e.g., increase in serum half-life, etc.). Such antibody conjugates are described in more detail below.

Amino Acid and Nucleic Acid Sequences

uPAR- and suPAR-binding agents can comprise a contiguous amino acid sequence that is at least 80% identical to (e.g., at least 85%, at least 90%, at least 95%, at least 98%, or 100%) to a contiguous sequence of any sequences listed below.

The present disclosure contemplates antibodies, and antigen-binding fragments thereof, comprising 1) a variable heavy chain (VH) polypeptide comprising VH complementarity determining regions (CDRs) of an antibody heavy chain variable region of a full-length VH amino acid sequence as described herein (e.g., 2G10, 2E9); and 2) a variable light chain (VL) polypeptide comprising VL complementarity determining regions (CDRs) of an antibody heavy chain variable region of a full-length VL amino acid sequence as described herein (e.g., 2G10, 2E9).

In certain embodiments, the complementarity determining regions (CDRs) of an anti-uPAR antibody described herein can include, without limitation, an antibody having the following CDRs:
a) CDRL1: light chain residues L26 to L32; CDRL2: light chain residues L50 to L52; CDRL3: light chain residues L91 to L96; CDRH1: heavy chain residues H26 to H32; CDRH2: heavy chain residues H53 to H55; and heavy chain residues H96 to H101, of an anti-uPAR antibody described herein such as antibody 2G10 (See, Chothia and Lesk, J. Mol. Biol. 196:901-917 (1987));
b) CDRL1: light chain residues L24 to L34; CDRL2: light chain residues L50 to L56; CDRL3: light chain residues L89 to L97; CDRH1: heavy chain residues H31 to H35b; CDRH2: heavy chain residues H50 to H65; and heavy chain residues H95 to H102, of an anti-uPAR antibody described herein such as antibody 2G10 (See, Kabat et al., Sequences of Proteins of Immunological Interest, 5th Ed. Public Health Service, National Institutes of Health, Bethesda, Md. (1991));
c) CDRL1: light chain residues L27c to L36; CDRL2: light chain residues L46 to L55; CDRL3: light chain residues L89 to L96; CDRH1: heavy chain residues H30 to H35b; CDRH2: heavy chain residues H47 to H58; and heavy chain residues H93 to H101, of an anti-uPAR antibody described herein such as antibody 2G10 (See, MacCallum et al., J. Mol. Biol. 262:732-745 (1996)); or
d) combinations of (a), (b), and/or (c), including CDRL2 residues L46 to L56; L47 to L56; L48 to L56; or L49 to L56; and/or CDRH2 residues H26 to H35; or H26 to H35b; and/or CHDRH3 residues H93 to H102; or H94 to H102, of an anti-uPAR antibody described herein such as antibody 2G10.

Accordingly, the CDRs of the monoclonal antibody 2G10 can be defined as in the table below.

| Heavy Chain | 2G10 |
|---|---|
| CDR1 | GDSVSSNSAAWN (SEQ ID NO: 3) |
| CDR2 | RTYYRSKWYND (SEQ ID NO: 4) |
| CDR3 | DPGGPLDDSFDI (SEQ ID NO: 5) |
| Light Chain | 2G10 |
| CDR1 | RSSQSLLRSNGYNYLD (SEQ ID NO: xx6 |
| CDR2 | LGSIRAS (SEQ ID NO: 7) |
| CDR2 | MQALQTPFT (SEQ ID NO: 8) |

Unless otherwise indicated antibody light and heavy chain variable sequences, including variable domain and framework regions are numbered according to Kabat et. al. supra.

Amino acid sequences of the full-length variable heavy chain (VH) and variable light chain (VL) of 2G10 are provided below. The underlining denotes the VH CDRs (from N to C-terminal direction, VH CDR1, VH CDR2, and VH CDR3, respectively) and the VL CDRs (from N to C-terminal direction, VL CDR1, VL CDR2, and VL CDR3, respectively) as defined using the Kabat system.

The VH and VL polypeptides of 2G10 are described below as having an amino acid sequence as provided below (the CDRs as defined above are underlined)

2G10 V_{H}:
(SEQ ID NO: 1)
QVQLQQSGPGLVKPSQTLSLTCAISGDSVSSNSAAWNWIRQSPSRGLEW
LGRTYYRSKWYNDYAVSVKSRIIINPDTSKNQFSLQLNSVTPEDTAVYY
CARDPGGPLDDSFDIWGQGTMVTVSSASTKGPSVFPLAPSSKSTSGGTA
ALGCLVKDYFPEPVTVSWNSGALTSGVHTFPAVLQSSGLYSLSSVVTVP
SSSLGTQTYICNVNHKPSNTKVDKKVEPKSC
2G10 V_{L}:
(SEQ ID NO: 2)
MTQSPLSLPVTPGEPASISCRSSQSLLRSNGYNYLDWYLQKPGQSPQLL
IYLGSIRASGVPDRFSGSGSGTDFTLRISRVEAEDVGVYYCMQALQTPF
TFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYAC
EVTHQGLSSPVTKSFNRGEC

In some embodiments, the VH and VL polypeptides of 2G10 can be provided as having the following amino acid sequences:

2G10 V_{H}:
(SEQ ID NO: 10)
TDTLLLWVLLLWVPGSTGQVQLQQSGPGLVKPSQTLSLTCAISGDSVSS
NSAAWNWIRQSPSRGLEWLGRTYYRSKWYNDYAVSVKSRIIINPDTSKN
QFSLQLNSVTPEDTAVYYCARDPGGPLDDSFDIWGQGTMVTVSSASTKG
PSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSC
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAIEKTISKAKGQPREPQVYTLPPSRDELTKNQVSLTCL
VKGFYPSDIAVEWESNGQPENNYKTTPPVLDSDGSFFLYSKLTVDKSRW
QQGNVFSCSVMHEALHNHYTQKSLSLSPGK
2G10 V_{L}:
(SEQ ID NO: 2)
MTQSPLSLPVTPGEPASISCRSSQSLLRSNGYNYLDWYLQKPGQSPQLL
IYLGSIRASGVPDRFSGSGSGTDFTLRISRVEAEDVGVYYCMQALQTPF
TFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYAC
EVTHQGLSSPVTKSFNRGEC
In some embodiments, the VH and VL polypeptides
of 2G10 can be provided as having the following
amino acid sequences:
2G10 V_{H}:
(SEQ ID NO: 1)
QVQLQQSGPGLVKPSQTLSLTCAISGDSVSSNSAAWNWIRQSPSRGLEW
LGRTYYRSKWYNDYAVSVKSRIIINPDTSKNQFSLQLNSVTPEDTAVYY
CARDPGGPLDDSFDIWGQGTMVTVSSASTKGPSVFPLAPSSKSTSGGTA
ALGCLVKDYFPEPVTVSWNSGALTSGVHTFPAVLQSSGLYSLSSVVTVP
SSSLGTQTYICNVNHKPSNTKVDKKVEPKSC
2G10 V_{L}:
(SEQ ID NO: 11)
LDVVMTQSPLSLPVTPGEPASISCRSSQSLLRSNGYNYLDWYLQKPGQS
PQLLIYLGSIRASGVPDRFSGSGSGTDFTLRISRVEAEDVGVYYCMQAL
QTPFTFGQGTKLEIKRTVAAPSVFIFPPSDEQLKSGTASVVCLLNNFYP
REAKVQWKVDNALQSGNSQESVTEQDSKDSTYSLSSTLTLSKADYEKHK
VYACEVTHQGLSSPVTKSFNRGEC

Other antibodies of interest can include antibodies comprising the VH and VL CDRs of the monoclonal antibody 2E9. The VH and VL CDRs of 2E9 are provided below. The amino acid sequences of the full-length VH and VL of these monoclonal antibodies are provided below. The underlining denotes the VH CDRs (from N to C-terminal direction, VH CDR1, VH CDR2, and VH CDR3, respectively) and the VL CDRs (from N to C-terminal direction, VL CDR1, VL CDR2, and VL CDR3, respectively).

2E9 V_{H}:
(SEQ ID NO: 12)
QVQLQESGGGLVQPGGSLRLSCAASGFTFSSYAMSWVRQAPGKGLEWVS
AISGSGGSTYYADSVKGRFTISRDNSKNTLYLQMNSLRAEDTAVYYCAK
DEDYDYVWGSYRQYPSRYWGQGTLVTVSSASTKGPSVFPLAPSSKSTSG
GTAALGCLVKDYFPEPVTVSWNSGALTSGVHTFPAVLQSSGLYSLSSVV
TVPSSSLGTQTYICNVNHKPSNTKVDKKVEPKSC
2E9 V_{L}:
(SEQ ID NO: 13)
QSVLTQPPSVSVSPGQTASITCSGDNLGYKYASWYQQKPGQSPVLIIYQ
DKKRPSGIPERFSGSNSGNTATLTISGTQAMDEADYYCQAWDSSTSVVF
GGGTKLTVLGQPKAAPSVTLFPPSSEELQANKATLVCLISDFYPGAVTV
AWKADSSPVKAGVETTTPSKQSNNKYAASSYLSLTPEQWKSHRSYSCQV
THEGSTVEKTVAPTECS

Recombinant Antibody

The agents of the present disclosure may be an antibody produced by recombinant methods. Such antibodies can be produced by expression of a polynucleotide having a nucleotide sequence encoding a polypeptide that is at least 80% identical to (e.g., at least 85%, at least 90%, at least 95%, at least 98%) to a contiguous sequence of any sequence listed above. The percent identity of nucleic acids is based on the shorter of the sequences compared. Well known programs such as BLASTN (2.0.8) (Altschul et al. (1997) Nucl. Acids. Res. 25:3389-3402) using default parameters and no filter may be employed to make a sequence comparison. Examples of nucleic acids encoding the antibodies of the present disclosure are discussed later below.

Methods for producing recombinant antibodies are known in the art. For example, the nucleic acids encoding the antibody, or at least a CDR of a heavy chain polypeptide or at least a CDR of a light chain polypeptide, are introduced directly into a host cell, and the cell incubated under conditions sufficient to induce expression of the encoded antibody. The recombinant antibody may be glycosylated by an endogenous glycosyl-transferase in the host cells, unglycosylated, or may have an altered glycosylation pattern.

Recombinant antibodies include chimeric antibodies. Chimeric antibodies are immunoglobulin molecules comprising human and non-human portions. More specifically, the antigen combining region (or variable region) of a humanized chimeric antibody is derived from a non-human source (e.g. murine), and the constant region of the chimeric antibody (which confers biological effector function to the immunoglobulin) is derived from a human source. The chimeric antibody can have the antigen binding specificity of the non-human antibody molecule and the effector function conferred by the human antibody molecule. A large number of methods of generating chimeric antibodies are well known to those of skill in the art. An alternative approach is the generation of humanized antibodies by linking the CDR regions of non-human antibodies to human constant regions by recombinant DNA techniques. See Queen et al., Proc. Natl. Acad. Sci. USA 86: 10029-10033 (1989).

Human Antibodies

The uPAR- and suPAR-binding agents of the present disclosure may be a fully human antibody. Human antibodies are primarily composed of characteristically human polypeptide sequences. A subject human antibody can be produced by a wide variety of methods (see, e.g., Larrick et al., U.S. Pat. No. 5,001,065). Human antibodies may be derived from a fully human Fab phage display library, as described in de Haard et al. (1999) Journal of Biological Chemistry. 274, 18218-18230

Human antibodies can also be produced initially in trioma cells (descended from three cells, two human and one mouse). Genes encoding the antibodies are then cloned and expressed in other cells, particularly non-human mammalian cells. The general approach for producing human antibodies by trioma technology has been described by Ostberg et al. Hybridoma 1983, 2: 361-367, Ostberg, U.S. Pat. No. 4,634,664, and Engelman et al., U.S. Pat. No. 4,634,666. Triomas have been found to produce antibody more stably than ordinary hybridomas made from human cells.

Accordingly, the present disclosure contemplates a DNA molecule comprising a nucleic acid sequence encoding an antibody that binds to uPAR and suPAR (e.g. a nucleic acid encoding 2G10). Nucleic acid sequences will be described later below.

Conjugates

uPAR- and suPAR binding agents of the present disclosure can be modified by chemical conjugation to a moiety of interest. For example, an agent may be conjugated to a second molecule of a different type (e.g. nucleic acid to a non-nucleic acid, or a peptide to a non-peptide). Where the agent is an antibody, the antibody conjugated to a second molecule is referred to as an “antibody conjugate.” The compositions containing the agents can encompass aggregates of conjugates, as they are readily taken up by cells.

Conjugated agents retain a desired activity, while exploiting properties of the second molecule of the conjugate to impart an additional desired characteristic. For example, a subject agent (e.g. antibody) can be conjugated to a second molecule that aids in solubility, storage or other handling properties, half-life, and/or controls release. Other examples include the conjugation of a dye, fluorophore or other detectable labels or reporter molecules for assays, tracking and the like. More specifically, a subject antibody can be conjugated to a second molecule such as a peptide, polypeptide, dye, fluorophore, luciferase, nucleic acid, carbohydrate, lipid and the like, such as the attachment of a lipid moiety, including N-fatty acyl groups such as N-lauroyl, N-oleoyl, fatty amines such as dodecyl amine, oleoyl amine, and the like.

The present disclosure further provides a conjugated agent that comprises a moiety that modifies cellular uptake relative to unconjugated material. The conjugate may exhibit increased cellular uptake relative to unconjugated material. In alternative embodiments, the conjugate exhibits decreased cellular uptake relative to unconjugated material. In this aspect, the efficiency of cellular uptake can be increased or decreased by linking to small organic or inorganic molecules, polymers, peptides or proteins that facilitate, or inhibit endocytosis. For example, a given antibody can be linked to a ligand for a target receptor or large molecule that is more easily engulfed by endocytotic mechanisms, such as another antibody. The antibody or other ligand can then be internalized by endocytosis and the payload released by acid hydrolysis or enzymatic activity when the endocytotic vesicle fuses with lysosomes. As such, the conjugate may be one that increases endocytosis relative to unconjugated agent. To decrease cellular uptake, the conjugate can include a ligand that retains the antibody on the surface of a cell, which can be useful as a control for cellular uptake, or in some instances decrease uptake in one cell type while increasing it in others.

uPAR- and suPAR-binding agents may also be detectably labeled, either directly or indirectly. Direct labels include radioisotopes (e.g., ¹²⁵I; ³⁵S, ¹¹¹In, ^{99m}Tc, and the like); enzymes whose products generate a signal (e.g., luciferase, β-galactosidase, horse radish peroxidase, alkaline phosphatase, and the like); fluorescent labels (e.g., fluorescein isothiocyanate, rhodamine, phycoerythrin, and the like); fluorescence emitting metals, e.g., ¹²⁵Eu, or others of the lanthanide series, attached to the antibody through metal chelating groups such as EDTA; chemiluminescent compounds, e.g., luminol, isoluminol, acridinium salts, and the like; bioluminescent compounds, e.g., luciferin; fluorescent proteins; or MRI contrast agents and the like. Indirect labels include second antibodies specific for a subject antibody, wherein the second antibody is labeled as described above; and members of specific binding pairs, e.g., biotin-avidin, and the like.

### Pharmaceutical Compositions

The present disclosure provides pharmaceutical compositions with a uPAR/suPAR-binding agent (e.g. antibody).

Pharmaceutical compositions can include a pharmaceutically acceptable excipient, which can be a solution such as an aqueous solution (e.g., a saline solution). The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like.

An antibody of the present disclosure can be formulated for parenteral administration for use in the methods described below. Where an antibody is administered as a liquid injectable (such as in those embodiments where they are administered intravenously, intraarterially, or directly into a tissue), an antibody formulation may be provided as a ready-to-use dosage form, or as a reconstitutable storage-stable powder or liquid composed of pharmaceutically acceptable carriers and excipients.

Pharmaceutical compositions can also contain one or more of: a salt, e.g., NaCl, MgCl, KCl, MgSO₄, etc.; a buffering agent, e.g., a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), N-tris[Hydroxymethyl]methyl-3-aminopropanesulfonic acid (TAPS), etc.; a solubilizing agent; a detergent, e.g., a non-ionic detergent such as Tween-20, etc.; a protease inhibitor; glycerol; and the like.

The concentration of antibody in the pharmaceutical formulations can vary from less than about 0.10%, usually at or at least about 2% to as much as 20% to 50% or more by weight, and will be selected primarily by fluid volumes, viscosities, etc., in accordance with the particular mode of administration selected and the patient's needs. The resulting compositions may be in the form of a solution, suspension, tablet, pill, capsule, powder, gel, cream, lotion, ointment, aerosol or the like.

Compositions of the present disclosure can include a therapeutically effective amount of a subject agent (e.g. antibody), as well as any other compatible components, as needed. By “therapeutically effective amount” is meant that the administration of that amount to an individual, either in a single dose, as part of a series of the same or different antibody or compositions, is effective to provide a desired effect (e.g., inhibition of podocyte depolarization). The therapeutically effective amount can be adjusted in connection with dosing regimen and diagnostic analysis of the subject's condition (e.g., monitoring for the present or absence of a suPAR in a sample from the subject using, for example, an anti-uPAR/suPAR antibody of the present disclosure.

The compositions of the present disclosure encompass those that contain more than one type of agents (e.g. antibodies). The composition may contain at least two, at least three, at least four or more different types of agents (e.g. antibodies). Where the agents in the subject compositions are antibodies, the antibodies may differ in their amino acid sequence, modification by conjugation, affinity, epitopes of uPAR bound, and/or effects on uPAR/suPAR ligand binding. For example, the composition may contain a first antibody that competes with integrins (e.g. β1 integrins) binding to uPAR and a second antibody that competes with urokinase for binding to uPAR. Alternatively, a composition may contain a first antibody that binds to uPAR and competes with urokinase binding to uPAR and a second antibody that binds to uPAR and competes with urokinase binding to uPAR and does or does not compete with the binding of the first antibody.

Other agents that may be included in the subject compositions include agents useful for treating a condition. For example, combination therapies discussed later below may use subject compositions containing one or more drug in addition to the one or more subject antibodies. Such additional agents may be provided in the same pharmaceutical composition, or provided as a separate pahramceutical composition in a kit with a pharmaceutical composition containing an anti-uPAR/suPAR antibody fo the present disclosure.

The amount of composition administered to an animal, e.g., a human, in the context of the present disclosure should be sufficient to effect a prophylactic or therapeutic response in the animal over a reasonable time frame, and varies depending upon the goal of the administration, the health and physical condition of the individual to be treated, age, the degree of resolution desired, the formulation of the antibody composition, the treating clinician's assessment of the medical situation, and other relevant factors. One skilled in the art will also recognize that dosage will depend on a variety of factors including the strength of the particular compound employed, the condition of the animal, and the body weight of the animal, as well as the severity of the illness and the stage of the disease. The size of the dose will also be determined by the existence, nature, and extent of any adverse side-effects that might accompany the administration of a particular compound. Thus it is expected that the amount will fall in a relatively broad range, but can nevertheless be routinely determined through various features of the subject such as those features noted above.

A suitable treatment regimen may be a single dose schedule or a multiple dose schedule (e.g., including ramp and maintenance doses). Where the subject is a kidney transplant patient, the anti-uPAR/suPAR antibody can be administered prior to transplant surgery, after transplant surgery or after recurrent of FSGS.

As indicated below, a subject composition may be administered in conjunction with other agents, and thus doses and regiments can vary in this context as well to suit the needs of the subject.

### Methods of Production

Wherein the agent is an antibody, the antibodies can be prepared using a wide variety of techniques known in the art including the use of hybridoma, recombinant, and phage display technologies, or a combination thereof. For example, antibody may be made from E. coli or mammalian cells containing expression cassettes encoding whole antibodies or Fabs.

Anti-uPAR antibodies, including antigen binding fragments of anti-uPAR antibodies, may also be produced by genetic engineering. Where the protein is produced using recombinant techniques, the proteins may be produced as an intracellular protein or as an secreted protein, using any suitable construct and any suitable host cell, which can be a prokaryotic or eukaryotic cell, such as for example a bacterial (e.g. E. coli) or a yeast host cell, respectively.

Examples of eukaryotic cells that may be used as host cells include yeast cells, insect cells, mammalian cells, and/or plant cells. Where mammalian host cells are used, the cells may include one or more of the following: human cells (e.g. Hela, 293, H9 and Jurkat cells); mouse cells (e.g., NIH3T3, L cells, and C127 cells); primate cells (e.g. Cos 1, Cos 7 and CV1) and hamster cells (e.g., Chinese hamster ovary (CHO) cells).

Vectors, each containing one heavy chain gene and one light chain gene retaining the initial antigen specificity, may be produced by insertion of appropriate sections of the nucleic acids encoding the antibodies into the expression vectors. A library of clones which co-express a heavy and light chain (comprising for example an intact antibody, an Fab fragment or an antigen binding fragment of an antibody molecule) can also be generated. The vectors that carry these genes may be co-transfected into a host (e.g. bacteria, insect cells, mammalian cells, or other suitable protein production host cell). Alternatively, the heavy and light chain may be inserted into a single vector and transfected into a host (e.g. bacteria, insect cells, mammalian cells, or other suitable protein production host cell).

Methods for introduction of genetic material into host cells include, for example, transformation, electroporation, conjugation, calcium phosphate methods, cationic peptide-based methods, polyethyleneimine-based methods, and the like. The method for transfer can be selected so as to provide for stable expression of the introduced antibody-encoding nucleic acid, such as by for example allowing selection for an antibiotic resistance marker (e.g. using gentimycin, ampicillin, kanamycin, G418 and the like), or a metabolism marker (e.g. selection for glutamine synthesis in glutamine free medium with or without methionine sulfoximine, or selection for DHFR with or without methotrexate). The antibody-encoding nucleic acid can be provided as an inheritable episomal element (e.g., plasmid) or can be genomically integrated. A variety of appropriate vectors for use in production of an antibody of interest are available commercially. When antibody gene synthesis is induced in the transfected host, the heavy and light chain proteins self-assemble to produce active antibodies that can be detected by assaying binding with the antigen or immunogen and isolated using techniques known in the art.

Further examples of techniques which can be used to produce single-chain Fvs and other antibodies include those described in Huston et al., Methods in Enzymology 1991, 203:46-88; and Skerra et al. (1988) Science 240:1038-1040. Antibodies can be humanized using a variety of techniques known in the art, veneering or resurfacing, and chain shuffling. Isolation and purification of antibodies can be accomplished using techniques known in the art, and can provide for antibody-containing preparations at least 50% to 60%, by weight, free from organic molecules with which the antibody is naturally associated or with which it is associated during manufacture.

Nucleic Acid

The present disclosure contemplates cells expressing a uPAR antibody or suPAR antibody as disclosed herein, e.g., by expression of heavy and light chain-encoding, or heavy and light chain fragment encoding, expression cassettes. Examples of encoding nucleic acids include a nucleic acid encoding a polypeptide comprising one or more CDRs at least about 85%, 90%, 95%, 98%, 99%, or 100% identical to those CDRs depicted herein. In another example, the antibody has one or more light and heavy chain complementarity determining region (CDR) polypeptide sequences at least about 85%, 90%, 95%, 98%, 99%, or 100% identical to those light and heavy chain CDR polypeptide sequences depicted in herein.

The disclosure further contemplates recombinant host cells containing an exogenous polynucleotide encoding at least a CDR of a heavy chain polypeptide or at least a CDR of a light chain polypeptide of the subject antibody.

The present disclosure thus contemplates nucleic acid (e.g., DNA) encoding a VH and/or VL polypeptide having the CDRs of 2G10 or 2E9, as well as nucleic acid (e.g., DNA) encoding a VH polypeptide and/or VL polypeptide of 2G10 or 2E9. The nucleic acid can comprise a contiguous nucleic acid sequence that is at least 80% identical to (e.g., at least 85%, at least 90%, at least 95%, at least 98%, or 100%) to a contiguous sequence of any sequences listed below.

2G10 V_{H}:
(SEQ ID NO: 14)
CAGGTACAGCTGCAGCAGTCAGGTCCAGGACTGGTGAAGCCCTCGCAGA
CCCTCTCACTCACCTGTGCCATCTCCGGGGACAGTGTCTCTAGCAACAG
TGCTGCTTGGAACTGGATCAGGCAGTCCCCATCGAGAGGCCTTGAGTGG
CTGGGAAGGACATACTACAGGTCCAAGTGGTATAATGATTATGCAGTAT
CCGTGAAAAGTCGAATAATTATCAACCCAGACACATCCAAGAACCAGTT
CTCCCTGCAGCTGAACTCTGTGACTCCCGAGGACACGGCTGTGTATTAC
TGTGCAAGAGATCCGGGGGGGCCTCTCGATGATAGTTTTGATATCTGGG
GCCAAGGGACAATGGTCACCGTCTCAAGCGCCTCCACCAAGGGCCCATC
GGTCTTCCCCCTGGCACCCTCCTCCAAGAGCACCTCTGGGGGCACAGCG
GCCCTGGGCTGCCTGGTCAAGGACTACTTCCCCGAACCGGTGACGGTGT
CGTGGAACTCAGGCGCCCTGACCAGCGGCGTCCACACCTTCCCGGCTGT
CCTACAGTCCTCAGGACTCTACTCCCTCAGCAGCGTAGTGACCGTGCCC
TCCAGCAGCTTGGGCACCCAGACCTACATCTGCAACGTGAATCACAAGC
CCAGCAACACCAAGGTGGACAAGAAAGTTGAGCCCAAATCTTGT
2G10 V_{L}:
(SEQ ID NO: 15)
CTTGATGTTGTGATGACTCAGTCTCCACTCTCCCTGCCCGTCACCCCTG
GAGAGCCGGCCTCCATCTCCTGCAGGTCTAGTCAGAGCCTCCTGCGTAG
TAATGGATACAACTATTTGGATTGGTACCTGCAGAAGCCAGGGCAGTCT
CCACAGCTCCTGATCTATTTGGGTTCTATTCGGGCCTCCGGGGTCCCTG
ACAGGTTCAGTGGCAGTGGATCGGGCACAGATTTTACACTGAGAATTAG
CAGAGTGGAGGCTGAGGATGTTGGGGTTTATTACTGCATGCAAGCTCTA
CAAACCCCGTTCACTTTTGGCCAGGGGACCAAGCTGGAGATCAAGCGAA
CTGTGGCTGCACCATCTGTCTTCATCTTCCCGCCATCTGATGAGCAGTT
GAAATCTGGAACTGCCTCTGTTGTGTGCCTGCTGAATAACTTCTATCCC
AGAGAGGCCAAAGTACAGTGGAAGGTGGATAACGCCCTCCAATCGGGTA
ACTCCCAGGAGAGTGTCACAGAGCAGGACAGCAAGGACAGCACCTACAG
CCTCAGCAGCACCCTGACGCTGAGCAAAGCAGACTACGAGAAACACAAA
GTCTACGCCTGCGAAGTCACCCATCAGGGCCTGAGCTCGCCCGTCACAA
AGAGCTTCAACAGGGGAGAGTGT
2E9 V_{H}:
(SEQ ID NO: 16)
CAGGTGCAGCTGCAGGAGTCGGGGGGAGGCTTGGTACAGCCTGGGGGGT
CCCTGAGACTCTCCTGTGCAGCCTCTGGATTCACCTTTAGCAGCTATGC
CATGAGCTGGGTCCGCCAGGCTCCAGGGAAGGGGCTGGAGTGGGTCTCA
GCTATTAGTGGTAGTGGTGGTAGCACATACTACGCAGACTCCGTGAAGG
GCCGGTTCACCATCTCCAGAGACAATTCCAAGAACACGCTGTATCTGCA
AATGAACAGCCTGAGAGCCGAGGACACGGCCGTATATTACTGTGCGAAA
GATGAGGATTATGATTACGTTTGGGGGAGTTATCGACAATACCCCAGTC
GCTACTGGGGCCAGGGAACCCTGGTCACCGTCTCAAGCGCCTCCACCAA
GGGCCCATCGGTCTTCCCCCTGGCACCCTCCTCCAAGAGCACCTCTGGG
GGCACAGCGGCCCTGGGCTGCCTGGTCAAGGACTACTTCCCCGAACCGG
TGACGGTGTCGTGGAACTCAGGCGCCCTGACCAGCGGCGTCCACACCTT
CCCGGCTGTCCTACAGTCCTCAGGACTCTACTCCCTCAGCAGCGTAGTG
ACCGTGCCCTCCAGCAGCTTGGGCACCCAGACCTACATCTGCAACGTGA
ATCACAAGCCCAGCAACACCAAGGTGGACAAGAAAGTTGAGCCCAAATC
TTGT
2E9 V_{L}:
(SEQ ID NO: 17)
CAGTCTGTGCTGACTCAGCCACCCTCAGTGTCCGTGTCCCCAGGACAGA
CAGCCAGCATCACCTGCTCTGGAGATAATTTGGGGTATAAATATGCTTC
CTGGTATCAGCAGAAGCCAGGCCAGTCCCCTGTGCTGATCATCTATCAA
GATAAGAAGCGGCCCTCTGGGATCCCTGAGCGATTCTCTGGCTCCAACT
CTGGGAACACAGCCACTCTGACCATCAGCGGGACCCAGGCTATGGATGA
GGCTGACTATTACTGTCAGGCGTGGGACAGCAGCACTTCTGTGGTATTC
GGCGGAGGGACCAAGCTGACCGTCCTAGGTCAGCCCAAGGCTGCCCCCT
CGGTCACTCTGTTCCCACCCTCCTCTGAGGAGCTTCAAGCCAACAAGGC
CACACTGGTGTGTCTCATAAGTGACTTCTACCCGGGAGCCGTGACAGTG
GCCTGGAAGGCAGATAGCAGCCCCGTCAAGGCGGGAGTGGAGACCACCA
CACCCTCCAAACAAAGCAACAACAAGTACGCGGCCAGCAGCTATCTGAG
CCTGACGCCTGAGCAGTGGAAGTCCCACAGAAGCTACAGCTGCCAGGTC
ACGCATGAAGGGAGCACCGTGGAGAAGACAGTGGCCCCTACAGAATGTT
CA

### Diagnostic Methods

The present disclosure provides methods of detecting suPAR in a biological sample in situ or isolated from a subject. Such detection of suPAR can facilitate identification of patients who can benefit from anti-uPAR/anti-suPAR antibody therapy, which can include patients with FSGS, including, without limitation, kidney transplant patients who are at risk of development recurrence FSGS. Kidney transplant patients with native kidney FSGS are at risk of recurrent FSGS. [NOTE: Correct?] Such diagnostics can be useful to identify patients amenable to the therapies disclosed herein, and/or to monitor response to therapy.

The subject diagnostic method generally involves contacting a sample containing a cell with a subject agent (e.g. antibody); and directly or indirectly detecting binding of the subject agent (e.g. antibody) to suPAR in the sample. The biological sample obtained from a patient suspected of having, or known to have FSGS, a patient undergoing treatment, or a patient being tested for susceptibility to treatment using an anti-uPAR/suPAR antibody of the present disclosure.

The biological sample from the subject can be any sample in which suPAR may be present, including but not limited to body fluids including blood or fractions thereof (e.g., plasma, serum) or urine.

Assays can take a wide variety of forms, such as competition, direct reaction, or sandwich type assays. Examples of assays include Western blots; agglutination tests; enzyme-labeled and mediated immunoassays, such as enzyme-linked immunosorbent assays (ELISAs); biotin/avidin type assays; radioimmunoassays; immunoelectrophoresis; immunoprecipitation, fluorescence activated cell sorting, and the like. The reactions generally include detectable labels such as fluorescent, chemiluminescent, radioactive, enzymatic labels or dye molecules, or other methods for detecting the formation of a complex between antigen in the sample and the antibody reacted therewith.

The assays can involve separation of unbound antibody in a liquid phase from a solid phase support to which antigen-antibody complexes are bound. Solid supports which can be used include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, and the like.

Where a solid support is used, the solid support is usually first reacted with a solid phase component (e.g., an anti-suPAR antibody) under suitable binding conditions such that the component is sufficiently immobilized to the support. Sometimes, immobilization to the support can be enhanced by first coupling the antibody to a protein with better binding properties, or that provides for immobilization of the antibody on the support with out significant loss of antibody binding activity or specificity. Suitable coupling proteins include, but are not limited to, macromolecules such as serum albumins including bovine serum albumin (BSA), keyhole limpet hemocyanin, immunoglobulin molecules, thyroglobulin, ovalbumin, and other proteins well known to those skilled in the art. Other molecules that can be used to bind antibodies to a support include polysaccharides, polylactic acids, polyglycolic acids, polymeric amino acids, amino acid copolymers, and the like, with the proviso that the molecule used to immobilize the antibody does not adversely impact the ability of the antibody to specifically bind antigen. Such molecules and methods of coupling these molecules to the antibodies are well known to those of ordinary skill in the art.

After reacting the solid support with the solid phase component, any non-immobilized solid-phase components are removed from the support by washing, and the support-bound component is then contacted with a biological sample suspected of containing suPAR under suitable binding conditions. After washing to remove any non-bound ligand, a secondary binder moiety is added under suitable binding conditions, wherein the secondary binder is capable of associating selectively with the bound ligand. The presence or absence of the secondary binder can then be detected using techniques well known in the art.

Alternatively, antibodies may be coupled to the beads non-covalently for example through contacting beads or other solid surface covalently attached to protein-A, protein-G, protein-L, or an antibody that recognizes the Fc region of one or more of the subject antibodies with one or more of the subject antibodies. The beads or other solid surface may then be contacted with the tissue, cell or extract to be tested, alternatively washed, collected (e.g. by centrifugation), and analyzed to determine the presence or absence of antibody-antigen complexes.

An ELISA method can be used, wherein the wells of a microtiter plate are coated with a subject anti-suPAR antibody. A biological sample containing or suspected of containing suPAR is then added to the coated wells. After a period of incubation sufficient to allow antibody binding, the plate(s) can be washed to remove unbound moieties and a detectably labeled secondary binding molecule added. The secondary binding molecule is allowed to react with any captured antigen, the plate washed and the presence or absence of the secondary binding molecule detected using methods well known in the art.

Where desired, the presence or absence of bound suPAR from a biological sample can be readily detected using a secondary binder comprising an antibody directed against the antibody ligands. For example, a number of anti-bovine, anti-rabbit, anti-equine, anti-rat, anti-mouse, and anti-human immunoglobulin (Ig) molecules are known in the art which can be readily conjugated to a detectable enzyme label, such as horseradish peroxidase, alkaline phosphatase or urease, using methods known to those of skill in the art. An appropriate enzyme substrate is then used to generate a detectable signal. In other related embodiments, competitive-type ELISA techniques can be practiced using methods known to those skilled in the art.

Assays can also be conducted in solution, such that the antibodies and suPAR form complexes under precipitating conditions. For example, the antibody can be attached to a solid phase particle (e.g., an agarose bead or the like) using coupling techniques known in the art, such as by direct chemical or indirect coupling. The antibody-coated particle is then contacted under suitable binding conditions with a biological sample suspected of containing suPAR to provide for formation of particle-antibody-suPAR complex aggregates which can be precipitated and separated from the sample using washing and/or centrifugation. The reaction mixture can be analyzed to determine the presence or absence of antibody-antigen complexes using any of a number of standard methods, such as those immunodiagnostic methods described above.

Assays can also be conducted in solution by fluorescence activated cell sorting FACS. For example, a biological sample known to comprise, or suspected of comprising, suPAR may be contacted with an antibody of the present invention. The subject antibody may be directly labeled (e.g. fluorescently labeled) or indirectly labeled (e.g. via a secondary antibody) as described herein or generally known in the art. The biological sample may then be counted, and in some cases sorted with a FACS machine. In some cases, fixed cells may be counted or sorted, in other cases, live cells may be counted or sorted.

The diagnostic assays described herein can be used to determine whether a subject has, or is at risk of developing, FSGS, which may be more or less amenable to therapy using antibody-based therapy as disclosed herein, as well as to monitor the progress of treatment in a subject. It also may be used to assess the course of other combination therapies. Thus, the diagnostic assays can inform selection of therapy and treatment regimen by a clinician.

suPAR can be detected by detection of specific binding of an antibody, e.g., a monoclonal antibody (mAb) that has the antigen-binding specificity of anti-uPAR/suPAR antibodies disclosed herein.

The above-described assay reagents, including the antibodies generated by immunization with uPAR according to the methods described previously, can be provided in kits, with suitable instructions and other necessary reagents, in order to conduct immunoassays as described above. The kit can also contain, depending on the particular immunoassay used, suitable labels and other packaged reagents and materials (i.e. wash buffers and the like). Standard immunoassays, such as those described above, can be conducted using these kits.

### Therapeutic Methods in Treatment of FSGS

The uPAR- and suPAR-binding agents (e.g. antibodies) of the present disclosure can find use in treatment of a subject having, or at risk of developing FSGS, are contemplated for the therapies and diagnostics described herein. Samples obtained from such subject are likewise suitable for use in the diagnostic methods of the present disclosure.

By “treatment” is meant that at least an amelioration of the symptoms associated with the condition afflicting the host is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g. terminated, such that the host no longer suffers from the condition, or at least the symptoms that characterize the condition. Thus treatment includes: (i) prevention, that is, reducing the risk of development of clinical symptoms, including causing the clinical symptoms not to develop, e.g., preventing disease progression to a harmful state; (ii) inhibition, that is, arresting the development or further development of clinical symptoms, e.g., mitigating or completely inhibiting an active disease, e.g., so as to decrease depolarization of podocytes and/or progressive sclerosis of glomeruli. Such treatment also includes situations where the pathological condition, or the progression of a pathological condition towards a more advanced disease state, or at least symptoms associated therewith, is reduced, or slowed down. In some cases, treatment includes situations wherein the mean time for survival between a patient population undergoing treatment comprising the administration of one or more subject antibodies and a control population not undergoing treatment is greater. In some cases, the increase in mean time for survival may be statistically significant.

A variety of hosts are treatable according to the methods. Generally such hosts are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the hosts will be humans.

### Focal Segmental Glomerulosclerosis (FSGS)

The kidney disease focal segmental glomerulosclerosis (FSGS) is characterized by development of scar tissue on the glomeruli. FSGS characterized by nephrotic range proteinuria and kidney dysfunction, can lead to kidney failure, which in turn requires treatment by either dialysis or kidney transplant. FSGS can be described in at least three different classifications: primary idiopathic FSGS, primary non-idiopathic FSGS, and secondary FSGS. The table below provides an overview of FSGS classifications, with causes (where known or suspected), candidate markers, and conventional therapy.

| FSGS | Primary | Primary non- | | |
|---|---|---|---|---|
| classification | idiopathic FSGS | idiopathic FSGS | | Secondary FSGS |
| Cause | Unknown; | Genetic | Viral infection | Reduced renal |
| | Characterized by | mutations in | causing | mass or other |
| | circulating | podocyte | podocyte | glomerular injury |
| | permeability | genes | dedifferentiation | |
| | factors and high | | | |
| | risk of FSGS | | | |
| | recurrence after | | | |
| | transplantation | | | |
| Candidate | suPAR, CD40, | CD2AP, | HIV, parvovirus | Hyperfiltration, |
| Markers | CLC-1 | Nephrin | | Obesity-associated |
| | | | | FSGS, drug |
| | | | | induced injury |
| Current | Calcineurin | Dexamethasone, Calcineurin | | Angiotensin |
| Treatment | inhibitors (CNI), | inhibitors | | blockers |
| | steroids, anti- | | | |
| | CD20 antibody | | | |
| | (e.g., rituximab), | | | |
| | plasma exchange | | | |

In view of the above, subjects contemplated for treatment in accordance with the anti-suPAR/uPAR antibodies and treatment methods of the present disclosure include those having, suspected of having, or at risk of having FSGS, including subjects having, suspected of having, or at risk of having primary idiopathic FSGS, having, suspected of having, or at risk of having primary non-idiopathic FSGS, or having, suspected of having, or at risk of having secondary FSGS, as well as subjects having, suspected of having, or at risk of having recurrent FSGS, including having, suspected of having, or at risk of having recurrent FSGS following kidney transplant. Subjects at risk of having or suspected of having FSGS include those in which analysis of candidate markers in the subject point toward or confirm an FSGS diagnosis (e.g., suPAR, CD40, CLC-1; CD2Ap, nehprin; viral infection (e.g., HIV, parvovirus); and/or hyperfiltration, obesity-associated FSGS, drug-induced injury.

Combination Therapies

The therapeutic methods described herein can include administration of a uPAR/suAPR agent (e.g. antibody) in combination with one or more other therapies. The combination therapy below can provide for additive or synergistic benefits relative to a regimen in which only one therapy is administered.

An example of combination therapy involves administering more than one type of agent (e.g. antibody) to a subject. As described above for pharmaceutical compositions, the therapeutic method may involve administering at least one, at least two, at least three or more different types of antibodies simultaneously or sequentially, including for example one or more subject antibodies. The antibodies may differ in the epitopes of uPAR or suPAR to which they bind. The method, for example, may involve administering antibodies from clone 2G10 and/or clone 2E9 to a subject in need of therapy. The antibodies may also bind the same or overlapping epitopes of uPAR and suPAR. The method for example may involve administering two or more antibodies that each inhibit the interaction between uPAR/suPAR and uPA, or two or more antibodies that each inhibit the interaction between uPAR/suPAR and an integrin, or two or more antibodies that each inhibit the interaction between uPAR/suPAR and vitronectin, or two or more antibodies that each inhibit the interaction between uPAR/suPAR and uPARAP, or any combination thereof.

Additional therapeutics that may or may not be administered in conjunction with a subject antibody, include but not limited to immunotherapy, chemotherapeutic agents, plasma exchange, and surgery (e.g., as those described further below). In addition, therapeutic administration of a subject antibody can also be post-therapeutic treatment of the subject with immunotherapy, chemotherapeutic agents, plasma exchange, and surgery.

For example, a subject antibody can be administered in combination with one or more of a calcineurin inhibitor (CNI), steroid (e.g., a corticosteroid, e.g., dexamethasone), angiotensin blockers, plasma exchange, and kidney transplant.

Where a combination therapy is administered, the therapy or treatment other than administration of antibody composition can be administered anywhere from simultaneously to up to 5 hours or more, e.g., 10 hours, 15 hours, 20 hours or more, prior to or after administration of a subject antibody. A subject antibody and other therapeutic intervention can be administered or applied sequentially, e.g., where a subject antibody is administered before or after another therapeutic treatment. Alternatively, a subject antibody and other therapy are administered simultaneously, e.g., where a subject antibody and a second therapy are administered at the same time, e.g., when the second therapy is a drug it can be administered along with a subject antibody as two separate formulations or combined into a single composition that is administered to the subject. Regardless of whether administered sequentially or simultaneously, as illustrated above, the treatments are considered to be administered together or in combination for purposes of the present disclosure.

Dosage

In the methods, an effective amount of an agent (e.g. a uPAR/suPAR antibody) is administered to a subject in need thereof. The amount administered can vary depending upon the goal of the administration, the health and physical condition of the individual to be treated, age, the degree of resolution desired, the formulation of a subject agent, the treating clinician's assessment of the medical situation, and other relevant factors. It is expected that the amount will fall in a relatively broad range that can be determined through routine trials. For example, the amount of subject agent employed to inhibit podocyte depolarization h is not more than about the amount that could otherwise be irreversibly toxic to the subject (i.e., maximum tolerated dose). In other cases the amount is around or even well below the toxic threshold, but still in an effective concentration range, or even as low as threshold dose.

Individual doses are typically not less than an amount required to produce a measurable effect on the subject, and may be determined based on the pharmacokinetics and pharmacology for absorption, distribution, metabolism, and excretion (“ADME”) of the antibody, and thus based on the disposition of the composition within the subject. This includes consideration of the route of administration as well as dosage amount, which can be adjusted for parenteral (applied by routes other than the digestive tract for systemic or local effects) applications, for example. For instance, administration of a subject antibody is typically via injection and often intravenous, intramuscular, intratumoral, intracranial, intraarterial, intraocular, intrathecal, or a combination thereof.

A uPAR/suPAR-binding agent (e.g. antibody) may be administered by infusion or by local injection. It also can be administered prior, at the time of, or after other therapeutic interventions, such as surgical intervention, e.g., kidney transplant. As noted above, a uPAR/suPAR antibody can also be administered as part of a combination therapy, in which at least one of an immunotherapy, a chemotherapy or a therapy involving plasma exchange and/or kidney transplant is administered to the subject.

As an example, the effective amount of a dose or dosing regimen can be gauged from the IC₅₀ of a given antibody for inhibiting or binding suPAR/uPAR. By “IC₅₀” is intended the concentration of a drug required for 50% inhibition in vitro. Alternatively, the effective amount can be gauged from the EC₅₀ of a given antibody concentration. By “EC₅₀” is intended the plasma concentration required for obtaining 50% of a maximum effect in vivo.

In general, with respect to the uPAR/suPAR-binding agents of the present disclosure, an effective amount is usually not more than 200× the calculated IC₅₀. Typically, the amount of an antibody that is administered is less than about 200×, less than about 150×, less then about 100× and many embodiments less than about 75×, less than about 60×, 50×, 45×, 40×, 35×, 30×, 25×, 20×, 15×, 10× and even less than about 8× or 2× the calculated IC₅₀. In one embodiment, the effective amount is about 1× to 50× of the calculated IC₅₀, and sometimes about 2× to 40×, about 3× to 30× or about 4× to 20× of the calculated IC₅₀. In other embodiments, the effective amount is the same as the calculated IC₅₀, and in certain embodiments the effective amount is an amount that is more than the calculated IC₅₀.

An effective amount may not be more than 100× the calculated EC₅₀. For instance, the amount of antibody that is administered is less than about 100×, less than about 50×, less than about 40×, 35×, 30×, or 25× and many embodiments less than about 20×, less than about 15× and even less than about 10×, 9×, 9×, 7×, 6×, 5×, 4×, 3×, 2× or 1× than the calculated EC₅₀. In one embodiment, the effective amount is about 1× to 30× of the calculated EC₅₀, and sometimes about 1× to 20×, or about 1× to 10× of the calculated EC₅₀. In other embodiments, the effective amount is the same as the calculated EC₅₀, and in certain embodiments the effective amount is an amount that is more than the calculated EC₅₀.

Effective amounts can readily be determined empirically from assays, from safety and escalation and dose range trials, individual clinician-patient relationships, as well as in vitro and in vivo assays such as those described herein and illustrated in the Experimental section, below.

The IC₅₀ may be calculated by inhibiting the agent binding to suPAR/uPAR (e.g. uPAR or suPAR alone or complexed with a ligand, such as integrins or uPA) in vitro. This aspect can be carried out by assessing the ability of the agent of interest to inhibit 2G10 antibody binding to uPAR or suPAR. In general, the procedure is carried out by standard ELISA in which the plates are coated with uPAR or suPAR at a concentration of about 1 μg/ml, and then processed and employed as described in the experimental examples to determine inhibition of antibody binding and the IC₅₀. These agents and others suitable for various aspects of this purpose can be employed.

Routes of Administration

In practicing the methods, routes of administration (path by which a subject agent is brought into a subject in need of therapy or diagnosis) may vary. A subject agent alone or in combinations described above can be administered systemically (e.g., by parenteral administration, e.g., by an intravenous route.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampoules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

The term “unit dosage form,” as used herein, refers to physically discrete units suitable as unitary dosages for human and animal subjects, each unit containing a predetermined quantity of compounds of the present disclosure calculated in an amount sufficient to produce the desired effect in association with a pharmaceutically acceptable diluent, carrier or vehicle. The specifications for the novel unit dosage forms depend on the particular compound employed and the effect to be achieved, and the pharmacodynamics associated with each compound in the host.

### Kits & Systems

Also provided are kits and systems that may find use in practicing the methods of treating FSGS, as described above. For example, kits and systems may include one or more of the compositions described herein, such as an anti-uPAR/suPAR antibody (e.g. 2G10), a nucleic acid encoding the same (especially a nucleic acid encoding a CDR of a heavy and/or light chain of 2G10), or a recombinant cell containing the same. Other optional components of the kit include: buffers, etc., for administering the anti-uPAR antibody, and/or for performing a diagnostic assay. The recombinant nucleic acids of the kit may also have restrictions sites, multiple cloning sites, primer sites, etc to facilitate their ligation to constant regions of non-2G10 encoding nucleic acids. The various components of the kit may be present in separate containers or certain compatible components may be precombined into a single container, as desired.

The kits and systems for practicing the methods may include one or more pharmaceutical formulations that include the antibody compositions described herein. As such, the kits may include a single pharmaceutical composition present as one or more unit dosages. In yet other embodiments, the kits may include two or more separate pharmaceutical compositions.

In addition to the above components, the kits may further include instructions for practicing the methods. These instructions may be present in the kits in a variety of forms, one or more of which may be present in or on the kit. One form in which these instructions may be present is as printed information on a suitable medium or substrate, e.g., a piece or pieces of paper on which the information is printed, in or on the packaging of the kit, in a package insert, etc. Yet another means would be a computer readable medium, e.g., diskette, CD, flash drive, thumb drive, etc., on which the information has been recorded. Yet another means that may be present is a website address which may be used via the internet to access the information at a removed site. Any convenient means may be present in the kits.

A kit may be provided for use in treating a host suffering from a cellular proliferative disease. This kit includes a pharmaceutical composition comprising antibody specific for uPAR/suPAR, and instructions for the effective use of the pharmaceutical composition in a method of treating a host having, suspected of having, or at risk of developing FSGS. Such instructions may include not only the appropriate handling properties, dosing regimen and method of administration, and the like, but can further include instructions to optionally screen the subject for suPAR/uPAR associated with the disease. This aspect can assist the practitioner of the kit in gauging the potential responsiveness of the subject to treatment with an antibody of the present disclosure, including timing and duration of treatment. Thus in another embodiment, the kit may further include an antibody or other reagent, such as 2G10, for detecting suPAR in a biological sample. The kit may also include an antibody that contains a conjugate with a detectable label, such as a fluorophore.

The following examples further illustrate the present invention and should not be construed as in any way limiting its scope.

### EXAMPLES

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Materials and Methods

The following methods and materials were used in the Examples below.

Human Podocyte Cell Model

The immortalized podocyte cell line was developed by transfection of primary human podocytes with the temperature sensitive SV40 T gene. These cells proliferate at the “permissive” temperature (33° C.) and are considered undifferentiated. After transferring to the “nonpermissive” temperature (37° C.), they enter growth arrest and by day 10-14 express markers of differentiated podocytes in vivo, such as nephrin, podocin, CD2 associated protein (CD2AP), synaptopodin, and known molecules of the slit diaphragm ZO-1, α, β, and γ-catenin, and P-cadherin.

Assay of Effect of Serum of Recurrent FSGS (rFSGS) Patients on Podocytes

Human podocytes as described above were cultured in RPMI medium supplemented with insulin, transferrin, selenium, sodium pyruvate (ITS-A, Gibco #513000), 10% FBS and penicillin/streptomycin. After differentiation for 14 days, cells were serum starved for 1h. Serum from rFSGS patients or from a control patient was added (4% final) and cell were cultured for additional 24h.

After fixation in PFA/sucrose, the actin cytoskeleton was visualized by labeling with rhodamine-conjugated phalloidin. DAPI was used for nuclei staining. Cells were imaged by confocal microscopy at 40× magnification and number of cells with intact stress fibers was counted.

To assess the effect of the fully humanized anti uPAR antibody 2G10, podocytes were cultured in the presence of 2G10 (1 ug/ml) or an IgG control antibody (1 ug/ml) prior to culturing with patient serum.

### Example 1 the Anti-Upar Antibody 2G10 Rescues Human Podocytes from the Effects of Rfsgs Patient Sera

Sera from three patients who exhibited recurrence of FSGS after kidney transplant and lost their grafts were used in this study. Serum was obtained from these patients prior to a further kidney transplant.

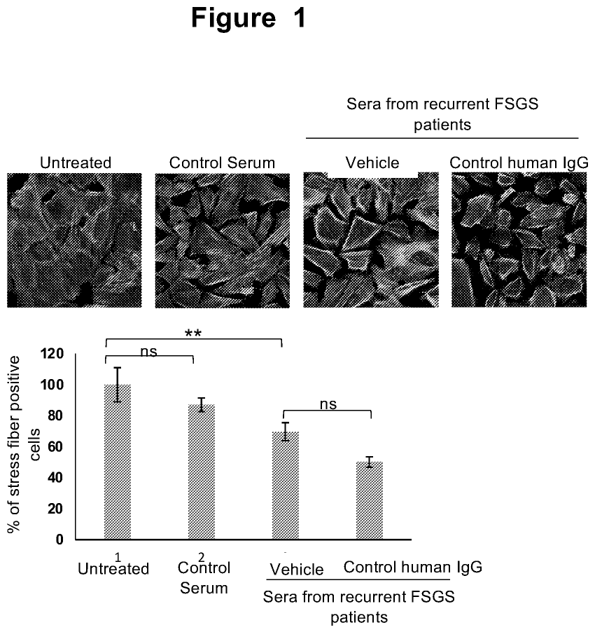

As shown in FIG. 1, culturing the human podocyte cell line as described in the materials and methods above in the presence of rFSGS patient sera causes significant depolarization of stress fibers as determined by number of stress fiber positive cells. These effects on human podocytes in this in vitro model are comparable to the abnormalities that occur in the kidney of transplanted patients with FSGS.

Control human IgG did not significantly impact the effect of rFSGS patient serum on human podocytes. In contrast, treatment of podocytes with control serum from a healthy did not cause any significant changes as compared to untreated podocytes.

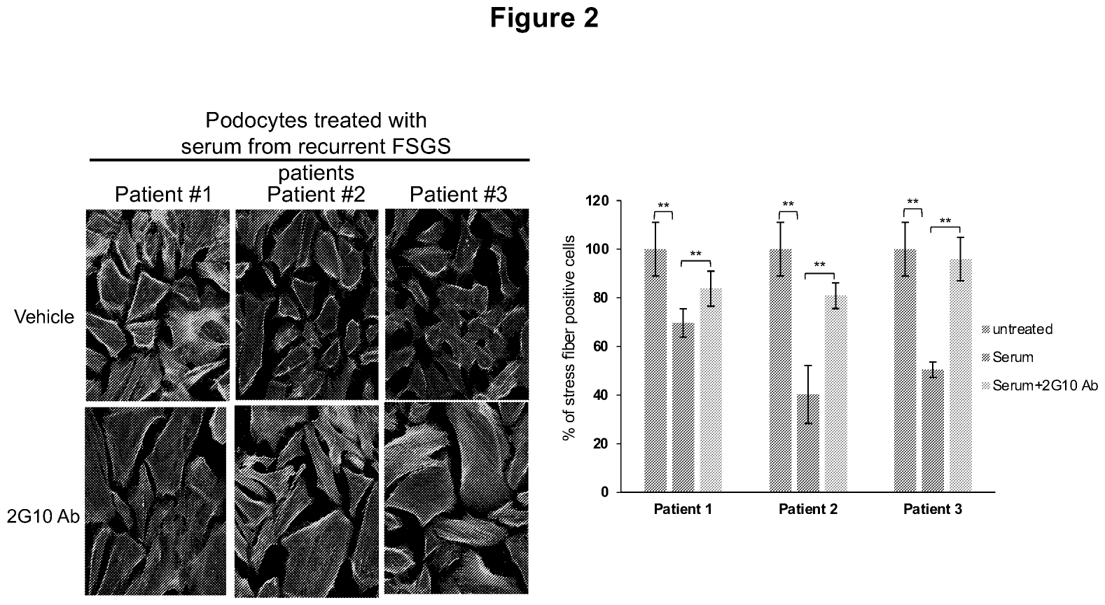

As shown in FIG. 2, sera from each of the rFSGS patients caused significant depolarization of stress fibers in the human podocyte cell model as determined by the number of stress fiber positive cells (30%, 59% and 49% reduction with respected to untreated podocytes respectively, FIG. 2, right panel). In contrast, 2G10 antibody rescued human podocyte stress fibers from the effect of rFSGS patient sera in each instance (FIG. 2, right panel, showing percentage of stress fiber positive cells for Patient 1, 2 and 3 cell when untreated (far left column in each patient sample), in the presence of rFSGS patient sera (middle column in each patient sample) and in the presence of rFSGS patient sera and 2G10 antibody (far right column in each patient sample).

The effect of a human anti-uPAR antibody on the pathogenic effect of samples from patients with recurrent FSGS on human podocytes has not been previously demonstrated. The 2G10 antibody has been previously shown to inhibit UPA and beta-integrin binding of the receptor uPAR. Without being held to theory, the effect of 2G10 is mediated by binding of 2G10 to soluble uPAR (suPAR) in patient serum.

The in vitro findings of the ability of 2G10 antibody to rescue human podocytes from the disrupting effect of the sera of patients with recurrent FSGS indicates that antibodies that block suPAR could be effective in preventing recurrence of FSGS.

### Example 2: Anti-Upar/Supar Antibody Therapy

The 2G10 antibody is administered by infusion to a patient with one or both native kidneys having or suspected of having FSGS so as to slow or present progression of disease to renal failure.

The 2G10 antibody is administered by infusion to a patient with a kidney transplant to mitigate or present recurrence of FSGS. Administration is prior to transplant, at the time of transplantation, or after transplant.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A method of treating or preventing focal segmental glomerulosclerosis (FSGS) in a subject, the method comprising:
administering to the subject an antibody, or antigen-binding fragment thereof, that competes for binding to uPAR with an antibody, or antigen-binding fragment thereof, comprising:
a variable heavy chain (VH) polypeptide comprising VH complementarity determining regions (CDRs) of an antibody heavy chain variable region comprising the amino acid sequence
(SEQ ID NO: 1)
QVQLQQSGPGLVKPSQTLSLTCAISGDSVSSNSAAWNWIRQSPSRGLEW
LGRTYYRSKWYNDYAVSVKSRIIINPDTSKNQFSLQLNSVTPEDTAVYY
CARDPGGPLDDSFDIWGQGTMVTVSSASTKGPSVFPLAPSSKSTSGGTA
ALGCLVKDYFPEPVTVSWNSGALTSGVHTFPAVLQSSGLYSLSSVVTVP
SSSLGTQTYICNVNHKPSNTKVDKKVEPKSC;
and
a variable light chain (VL) polypeptide comprising VL CDRs of an antibody light chain variable region comprising amino acid sequence
(SEQ ID NO: 2)
MTQSPLSLPVTPGEPASISCRSSQSLLRSNGYNYLDWYLQKPGQSPQLL
IYLGSIRASGVPDRFSGSGSGTDFTLRISRVEAEDVGVYYCMQALQTPF
TFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYAC
EVTHQGLSSPVTKSFNRGEC.

2. The method of claim 1, wherein the antibody, or antigen-binding fragment thereof, comprises:
a VH CDR1 comprising the amino acid sequence of GDSVSSNSAAWN (SEQ ID NO: 3);
a VH CDR2 comprising the amino acid sequence of RTYYRSKWYND (SEQ ID NO: 4)
a VH CDR3 comprising the amino acid sequence of DPGGPLDDSFDI (SEQ ID NO: 5);
a VL CDR1 comprising the amino acid sequence of RSSQSLLRSNGYNYLD (SEQ ID NO: 6);
a VL CDR2 comprising the amino acid sequence of LGSIRAS (SEQ ID NO: 7); and
a VL CDR3 comprising the amino acid sequence of MQALQTPFT (SEQ ID NO: 8).

3. The method of claim 2, wherein the antibody, or antigen-binding fragment thereof, comprises:
a) a heavy chain polypeptide comprising an amino acid sequence of at least 85% amino acid sequence identity to the full length V_{H} of 2G10; and
b) a light chain polypeptide comprising an amino acid sequence of at least 85% amino acid sequence identity to the full length V_{L} of 2G10.

4. The method of claim 1, wherein the subject is at risk of FSGS, and said administering is effective to prevent or ameliorate FSGS in the subject.

5. The method of claim 1, wherein the subject has or is suspected of having FSGS, and said administering is effective to treat FSGS in the subject.

6. The method of claim 1, wherein the subject is a candidate for kidney transplant or has undergone a kidney transplant.

7. The method of claim 1, wherein the subject has undergone a kidney transplant and is at risk of recurrent FSGS.

8. The method of claim 1, wherein the subject is a kidney transplant candidate and the antibody, or antigen-binding fragment thereof, is administered prior to kidney transplant.

9. The method of claim 1, wherein the subject has undergone kidney transplant, and the antibody, or antigen-binding fragment thereof, is administered at the time of kidney transplant.

10. The method of claim 1, wherein the subject has undergone kidney transplant, and the antibody, or antigen-binding fragment thereof, is administered following kidney transplant.

11. A method of inhibiting activity of urokinase-type plasminogen activator receptor (uPAR) and/or soluble uPAR (suPAR) in a subject having detectable blood level of suPAR, the method comprising:
administering to the subject an effective amount of an antibody, or antigen-binding fragment thereof, that specifically binds uPAR and suPAR, wherein the antibody, or antigen-binding fragment thereof, competes for binding to uPAR with an antibody, or antigen-binding fragment thereof, comprising:
a variable heavy chain (VH) polypeptide comprising VH complementarity determining regions (CDRs) of an antibody heavy chain variable region comprising the amino acid sequence
(SEQ ID NO: 1)
QVQLQQSGPGLVKPSQTLSLTCAISGDSVSSNSAAWNWIRQSPSRGLEW
LGRTYYRSKWYNDYAVSVKSRIIINPDTSKNQFSLQLNSVTPEDTAVYY
CARDPGGPLDDSFDIWGQGTMVTVSSASTKGPSVFPLAPSSKSTSGGTA
ALGCLVKDYFPEPVTVSWNSGALTSGVHTFPAVLQSSGLYSLSSVVTVP
SSSLGTQTYICNVNHKPSNTKVDKKVEPKSC;
and
a variable light chain (VL) polypeptide comprising VL CDRs of an antibody light chain variable region comprising amino acid sequence
(SEQ ID NO: 2)
MTQSPLSLPVTPGEPASISCRSSQSLLRSNGYNYLDWYLQKPGQSPQLL
IYLGSIRASGVPDRFSGSGSGTDFTLRISRVEAEDVGVYYCMQALQTPF
TFGQGTKLEIKRTVAAPSVFIFPPSDEQLKSGTASVVCLLNNFYPREAK
VQWKVDNALQSGNSQESVTEQDSKDSTYSLSSTLTLSKADYEKHKVYAC
EVTHQGLSSPVTKSFNRGEC.

12. The method of claim 11, wherein the antibody, or antigen-binding fragment thereof, comprises:
a VH CDR1 comprising the amino acid sequence of GDSVSSNSAAWN (SEQ ID NO: 3);
a VH CDR2 comprising the amino acid sequence of RTYYRSKWYND (SEQ ID NO: 4)
a VH CDR3 comprising the amino acid sequence of DPGGPLDDSFDI (SEQ ID NO: 5);
a VL CDR1 comprising the amino acid sequence of RSSQSLLRSNGYNYLD (SEQ ID NO: 6);
a VL CDR2 comprising the amino acid sequence of LGSIRAS (SEQ ID NO: 7); and
a VL CDR3 comprising the amino acid sequence of MQALQTPFT (SEQ ID NO: 8).

13. The method of claim 12, wherein the antibody, or antigen-binding fragment thereof, comprises:
a) a heavy chain polypeptide comprising an amino acid sequence of at least 85% amino acid sequence identity to the full length V_{H} of 2G10; and
b) a light chain polypeptide comprising an amino acid sequence of at least 85% amino acid sequence identity to the full length V_{L} of 2G10.

14. The method of claim 1, wherein the subject is at risk of FSGS, and said administering is effective to prevent or ameliorate FSGS in the subject.

15. The method of claim 1, wherein the subject has or is suspected of having FSGS, and said administering is effective to treat FSGS in the subject.

16. The method of claim 1, wherein the subject is a candidate for kidney transplant or has undergone a kidney transplant.

17. The method of claim 1, wherein the subject has undergone a kidney transplant and is at risk of recurrent FSGS.

18. The method of claim 1, wherein the subject is a kidney transplant candidate and the antibody, or antigen-binding fragment thereof, is administered prior to kidney transplant.

19. The method of claim 1, wherein the subject has undergone kidney transplant, and the antibody, or antigen-binding fragment thereof, is administered at the time of kidney transplant.

20. The method of claim 1, wherein the subject has undergone kidney transplant, and the antibody, or antigen-binding fragment thereof, is administered following kidney transplant.


SEQUENCE LISTING (17 sequences)

SEQ ID NO: 1 - protein, 227 aa, Homo sapiens
QVQLQQSGPGLVKPSQTLSLTCAISGDSVSSNSAAWNWIRQSPSRGLEWLGRTYYRSKWYNDYAVSVKSRIIINPDTSKNQFSLQLNSVTPEDTAVYYCARDPGGPLDDSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 2 - protein, 216 aa, Homo sapiens
MTQSPLSLPVTPGEPASISCRSSQSLLRSNGYNYLDWYLQKPGQSPQLLIYLGSIRASGVPDRFSGSGSGTDFTLRISRVEAEDVGVYYCMQALQTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 12 aa, Homo sapiens
GDSVSSNSAAWN

SEQ ID NO: 4 - protein, 11 aa, Homo sapiens
RTYYRSKWYND

SEQ ID NO: 5 - protein, 12 aa, Homo sapiens
DPGGPLDDSFDI

SEQ ID NO: 6 - protein, 16 aa, Homo sapiens
RSSQSLLRSNGYNYLD

SEQ ID NO: 7 - protein, 7 aa, Homo sapiens
LGSIRAS

SEQ ID NO: 8 - protein, 9 aa, Homo sapiens
MQALQTPFT

SEQ ID NO: 9 - protein, 281 aa, Homo sapiens
LRCMQCKTNGDCRVEECALGQDLCRTTIVRLWEEGEELELVEKSCTHSEKTNRTLSYRTGLKITSLTEVVCGLDLCNQGNSGRAVTYSRSRYLECISCGSSDMSCERGRHQSLQCRSPEEQCLDVVTHWIQEGEEGRPKDDRHLRGCGYLPGCPGSNGFHNNDTFHFLKCCNTTKCNEGPILELENLPQNGRQCYSCKGNSTHGCSSEETFLIDCRGPMNQCLVATGTHEPKNQSYMVRGCATASMCQHAHLGDAFSMNHIDVSCCTKSGCNHPDLDVQYR

SEQ ID NO: 10 - protein, 471 aa, Homo sapiens
TDTLLLWVLLLWVPGSTGQVQLQQSGPGLVKPSQTLSLTCAISGDSVSSNSAAWNWIRQSPSRGLEWLGRTYYRSKWYNDYAVSVKSRIIINPDTSKNQFSLQLNSVTPEDTAVYYCARDPGGPLDDSFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 220 aa, Homo sapiens
LDVVMTQSPLSLPVTPGEPASISCRSSQSLLRSNGYNYLDWYLQKPGQSPQLLIYLGSIRASGVPDRFSGSGSGTDFTLRISRVEAEDVGVYYCMQALQTPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 230 aa, Homo sapiens
QVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKDEDYDYVWGSYRQYPSRYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 13 - protein, 213 aa, Homo sapiens
QSVLTQPPSVSVSPGQTASITCSGDNLGYKYASWYQQKPGQSPVLIIYQDKKRPSGIPERFSGSNSGNTATLTISGTQAMDEADYYCQAWDSSTSVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 14 - DNA, 681 bp, Homo sapiens
caggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatccgtgaaaagtcgaataattatcaacccagacacatccaagaaccagttctccctgcagctgaactctgtgactcccgaggacacggctgtgtattactgtgcaagagatccgggggggcctctcgatgatagttttgatatctggggccaagggacaatggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 15 - DNA, 660 bp, Homo sapiens
cttgatgttgtgatgactcagtctccactctccctgcccgtcacccctggagagccggcctccatctcctgcaggtctagtcagagcctcctgcgtagtaatggatacaactatttggattggtacctgcagaagccagggcagtctccacagctcctgatctatttgggttctattcgggcctccggggtccctgacaggttcagtggcagtggatcgggcacagattttacactgagaattagcagagtggaggctgaggatgttggggtttattactgcatgcaagctctacaaaccccgttcacttttggccaggggaccaagctggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 16 - DNA, 690 bp, Homo sapiens
caggtgcagctgcaggagtcggggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatgaggattatgattacgtttgggggagttatcgacaataccccagtcgctactggggccagggaaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgt

SEQ ID NO: 17 - DNA, 639 bp, Homo sapiens
cagtctgtgctgactcagccaccctcagtgtccgtgtccccaggacagacagccagcatcacctgctctggagataatttggggtataaatatgcttcctggtatcagcagaagccaggccagtcccctgtgctgatcatctatcaagataagaagcggccctctgggatccctgagcgattctctggctccaactctgggaacacagccactctgaccatcagcgggacccaggctatggatgaggctgactattactgtcaggcgtgggacagcagcacttctgtggtattcggcggagggaccaagctgaccgtcctaggtcagcccaaggctgccccctcggtcactctgttcccaccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca